(19) United States
(12) Patent Application Publication
(10) Pub. No.: US 2024/0050341 A1
(43) Pub. Date: Feb. 15, 2024

(54) COMPOSITIONS AND METHODS FOR TREATING KERATIN FIBERS

(71) Applicant: L'Oreal, Paris (FR)

(72) Inventors: Cho-Cho KHINE, Scotch Plains, NJ (US); Ronak RUGHANI, Edison, NJ (US)

(21) Appl. No.: 18/231,729

(22) Filed: Aug. 8, 2023

Related U.S. Application Data
(60) Provisional application No. 63/396,181, filed on Aug. 8, 2022.

(30) Foreign Application Priority Data
Nov. 9, 2022 (FR) 2211644

(51) Int. Cl. A61K 8/365; A61Q 5/06; A61Q 5/00; A61K 8/73

(52) U.S. Cl. CPC A61K 8/365; A61Q 5/06; A61Q 5/002; A61K 8/738; A61K 2800/48; A61K 2800/5922

(57) ABSTRACT

The disclosure relates to compositions comprising (a) at least one carboxylic acid or salt thereof; (b) at least one cyclodextrin or derivative thereof; (c) at least one polysaccharide thickening agent; (d) at least one polyol; and (e) optionally water. The pH of the composition is less than 7. The compositions can be used for providing lasting curl definition and/or frizz and/or volume control to hair, even in high humidity. The disclosure also relates to methods of making and using the compositions, and kits containing the compositions.

### CROSS REFERENCE TO RELATED APPLICATIONS

This application claims the benefit of priority to U.S. Provisional Application No. 63/396,181, filed on Aug. 8, 2022, and FR Application No. 2211644, filed Nov. 9, 2022, both of which are incorporated by reference herein in their entireties.

### TECHNICAL FIELD

The present disclosure relates to compositions for treating or styling keratin fibers, such as curly or wavy hair, as well as methods of making and using the compositions, and kits comprising the compositions.

### BACKGROUND

Consumers with curly or wavy hair are often dissatisfied with the appearance of their hair. For example, curly hair may appear frizzy, with poor curl definition. Although products exist that are meant to reduce frizziness and/or improve curl definition, these products have drawbacks such as imparting a greasy feel and/or crunchy feel to the hair, weighing the hair down, fail to achieve smooth feel/touch, or are ineffective, particularly in humid climates.

Increasingly, consumers also seek hair products that have a natural look and feel, a light-weight feel, while imparting longer-lasting styling or shaping benefits to hair. Further, consumers seek products that offer multiple benefits, for example, combining frizz reduction and style hold with softening, elongation, or lengthening effects while still providing good curl definition. Moreover, consumers desire hair products that can protect hair from external factors such as high humidity which causes the hair to become very frizzy, unmanageable, and lose its shape and style or such as mechanical or physical or other external stresses on hair.

As such, there is a desire for new and improved products that can enhance the visual appearance of curliness of the hair, such as by improving the definition of the curls, preventing unwanted elongation of hair, and/or providing frizz and/or volume control to the hair.

The present disclosure relates to new compositions and methods of treating hair which will deliver both caring and styling/shaping benefits that are long lasting or durable and can be maintained overnight or when the hair is subjected to changes in the hair shape or style or configuration, and/or to disturbance of the hair fibers and/or to movement of the hair fibers as a result of sleeping or lying down and resting the head on a bed or other surface or the back part of a seat and/or as a result of wearing a head cap or cover. Such styling/shaping benefits include, for example, curl definition, frizz control, discipline, shape control/hold, volume control, integrity, durability, softness, smoothness, shine, natural feel, hydration, and/or light weight feel.

Compositions and methods according to the present disclosure can provide styling benefits that curly haired consumers desire on a daily basis, such as curl definition, moisture, conditioning, hold, frizz control, curl/shape retention, curl pick up, moisture to curls, and/or not leaving the curls feeling greasy or stiff, which benefits surprisingly last even through several shampoos.

### SUMMARY

The present disclosure relates to compositions for treating keratin fibers, such as curly or wavy hair, and in particular, relates to compositions for providing overall benefits to curly or wavy hair, such as maintaining and/or improving curl definition and hold, preventing unwanted elongation, providing shape control and discipline, providing frizz control and/or volume control, improving hair integrity, and/or preventing and reducing hair damage. The present disclosure also relates to methods of using the compositions to provide overall benefits described above to hair, particularly, curly or wavy hair.

It has now surprisingly been found that a combination of components disclosed herein can provide one or all of the benefits described above to keratin fibers, particularly curly or wavy hair. These benefits have been shown to be lasting, even in conditions of high humidity, and even after the hair is washed. In addition, the compositions are easy to apply and are environment friendly due to the use of natural materials.

In various embodiments, the compositions according to the present disclosure comprise (a) at least one carboxylic acid or salt thereof; (b) at least one cyclodextrin or derivative thereof; (c) at least one polysaccharide thickening agent; (d) at least one polyol; and (e) water. The pH of the composition is generally less than about 7. In various embodiments, the mole ratio of the (a) carboxylic acid(s) and salt(s) thereof to the (b) cyclodextrin(s) and derivative(s) thereof may range from about 0.5:1 to about 25:3, such as from about 4:1 to about 15:1, from about 5:1 to about 12:1, or from about 6:1 to about 10:1, or may be about 1:1, about 2:1, about 3:1, about 4:1, about 5:1, about 6:1, about 7:1, about 8:1, about 9:1, about 10:1, about 11:1, about 12:1, about 13:1, about 14:1, about 15:1, about 16:1, about 17:1, about 18:1, about 19:1, about 20:1, about 3:2, about 5:2, about 7:2, about 9:2, about 11:2, about 13:2, about 15:2, about 17:2, about 19:2, about 1:3, about 2:3, about 4:3, about 5:1, about 7:3, about 8:3, about 10:3, about 11:3, about 13:3, about 14:3, about 16:3, about 17:3, about 19:3, or about 20:3, including all ranges and subranges using any of the foregoing as upper or lower limits. In some embodiments, the weight ratio of the (a) carboxylic acid(s) and salt(s) thereof to the (b) cyclodextrin(s) and derivative(s) thereof may range from about 1:3 to about 6:1, for example may be about 1:3, about 1:2.5, about 1:2, about 1:1.5, about 1:1, about 1.5:1, about 2:1, about 2.5:1, about 3:1, about 3.5:1, about 4:1, about 4.5:1, about 5:1, about 5.5:1, or about 6:1, including all ranges and subranges using any of the foregoing as upper or lower limits. In various embodiments, the pH of the composition ranges from about 2 to about 6, such as from about 3 to about 5, from about 3.5 to about 5, from about 3 to about 4.5, from about 3.5 to about 4.5, or from about 3 to about 4. The compositions can be leave-in or rinse-off compositions.

In various embodiments, the (a) at least one carboxylic acid or salt thereof may be chosen from mono-carboxylic acids, di-carboxylic acids, tri-carboxylic acids, salts thereof, or mixtures thereof, such as, for example citric acid, lactic acid, malic acid, maleic acid, tartaric acid, glycolic acid, salts thereof, or mixtures thereof. In some further embodiments, suitable carboxylic acid(s) or salt(s) thereof may have a molecular weight of less than about 900 g/mol, preferably less than about 700 g/mol, less than about 500 g/mol, less than about 300 g/mol, or less than about 200 g/mol. In various embodiments, the total amount of the (a) carboxylic acids and salts thereof may range from about 0.1% to about 20%, preferably from about 0.1% to about 15%, from about 0.5% to about 15%, from about 0.5% to about 10%, from about 0.5% to about 5%, from about 1% to about 10%, or from about 1% to about 5% by weight, relative to the total weight of the composition.

In various embodiments, the (b) at least one cyclodextrin or derivative thereof may be chosen from compounds of formula:

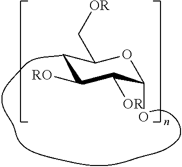

wherein R is chosen from H, CH₃, or a hydroxypropyl group, and n ranges from 6 to 8. For example, the at least one cyclodextrin or derivative thereof may be chosen from α-cyclodextrin, β-cyclodextrin, γ-cyclodextrin, methyl-α-cyclodextrin, methyl-β-cyclodextrin, methyl-γ-cyclodextrin, or mixtures thereof. The total amount of the (b) cyclodextrins and derivatives thereof may, in various embodiments, be present in an amount ranging from about 0.01% to about 20% by weight, such as, for example, from about 0.1% to about 18%, from about 0.5% to about 15%, from about 0.5% to about 10%, from about 0.5% to about 8%, from about 1% to about 13%, from about 1% to about 7%, or from about 1% to about 5% by weight, relative to the total weight of the composition.

In various embodiments, the (c) at least one polysaccharide thickening agent may be chosen from gums, for example gum arabic, tragacanth gum, karaya gum, guar gum, gellan gum, tara gum, locust bean gum, tamarind gum, xanthan gum, dehydroxanthan gum, Seneca gum, sclerotium gum, gellan gum, or the like. The total amount of polysaccharide thickening agent(s) may range from 0.05% to about 5%, preferably from about 0.1% to about 5%, more preferably from about 0.1% to about 3% by weight, relative to the total weight of the composition.

In various embodiments, the (d) at least one polyol may be chosen from ethylene glycol, propylene glycol, butylene glycol, hexylene glycol, pentylene glycol, diethylene glycol, dipropylene glycol, 1,3 propanediol, glycerin, polyethylene glycols, or combinations thereof. The total amount of (d) polyols may be at least 0.5%, for example may range from about 1% to about 10% by weight, relative to the total weight of the composition.

In various embodiments, water is present in an amount greater than 50%, for example ranging from about 70% to about 98%, from about 75% to about 98%, from about 80% to 98%, from about 85% to 98%, from about 90% to about 98%, from about 95% to about 98%, from about 70% to about 95%, from about 75% to about 95%, from about 80% to 95%, from about 85% to 95% by weight, relative to the total weight of the composition.

In further embodiments, the disclosure relates to compositions for treating curly hair, the compositions comprising (a) from about 0.1% to about 20% by weight of carboxylic acids chosen from citric acid and/or salts thereof; (b) from about 0.01% to about 20% by weight of cyclodextrins chosen from beta cyclodextrin and/or derivatives thereof; (c) from about 0.05% to about 5% by weight of one or more polysaccharide gums; (d) at least 0.5% by weight of polyols; and (e) at least 80% by weight of water, wherein the composition has a pH of less than 7.

The disclosure further relates to methods of making and using the compositions disclosed herein. The methods of using the compositions comprise applying the compositions according to the disclosure to the hair, and optionally, rinsing the compositions from the hair. In some embodiments, the methods may include shampooing and rinsing the hair before applying the composition to the hair; and/or conditioning and rinsing the hair after applying the composition to the hair. The hair treated with the compositions may be air dried or dried with a dryer, where the composition applied on the hair may be left on the hair or rinsed off before drying. In some embodiments, the methods further include styling the hair before the hair is dried, either while the composition is on the hair or after it has been rinsed off. The methods may impart one or more benefits such as maintaining and/or improving the curl definition and hold, providing volume control and/or frizz control, improving durability, improving the integrity to the hair, which may in various embodiments last for one or several hours, overnight, or for several days, even in high humidity condition and/or after the treated hair is washed.

The disclosure further relates to methods of treating hair comprising applying to the hair a composition disclosed herein; after leaving the composition on the hair for a desired period of time, such as 1 minutes to about 1 hour, optionally heating the hair at a temperature ranging from about 50° C. to about 225° C. for a desired period of time such as from about 1 minute to about 5 minutes; and optionally, rinsing the composition from the hair before or after heating the hair. After the composition is applied to the hair, in various embodiments, the hair may optionally be styled, e.g. by scrunching using hands before and/or while the hair is dried. In various embodiments, the hair may be heated by a heating appliances or apparatus such as a hooded dryer, a steamer, a blow dryer, or a flat iron. In further embodiments, the methods may comprise shampooing and rinsing the hair before applying the composition to the hair, and/or conditioning and rinsing the hair after heating the hair. In further embodiments, the methods may comprise applying a conditioner to the hair after applying the composition to the hair and before heating the hair, wherein the conditioner and the composition remain on the hair when the hair is heated; and optionally, rinsing the hair before or after heating the hair. In some embodiments, the methods may further comprise styling the hair after the hair is treated with the composition and before the hair is dried. For example, the hair may be scrunched to shape the style after the hair is heated and further rinsed. Styling is usually performed on wet or damp hair.

The disclosure further relates to kits comprising a first container containing a composition according to the present disclosure and/or a concentrated pre-mixture disclosed herein, and one or more additional containers containing one or more hair care/treatment compositions other than the compositions disclosed herein, such as, for example, a shampoo, a conditioner, a hair mask, or a hair styling composition. The pre-phase mixture, or stock mixture, is a concentrated combination of (a) at least one carboxylic acid or salt thereof; (b) at least one cyclodextrin or derivative thereof; (c) at least one polysaccharide thickening agent; and (d) at least one polyol. The pre-phase mixture can be diluted to provide a composition according to the disclosure near or at the time of use.

### BRIEF DESCRIPTION OF THE FIGURES

The accompanying drawings, which are incorporated herein and constitute part of this specification, illustrate exemplary embodiments of the disclosure, and, together with the general description given above and the description provided herein, serve to explain features of the disclosure.

FIG. 1 shows images of hair swatches treated with composition 1A according to an exemplary embodiment of the disclosure and comparative compositions C1, C2, and C3, and a control hair swatch treated with water, after 24 hours of exposure to a condition of 80% relative humidity (80% RH) at 27° C.

FIG. 2 is a graph showing frizz levels (frizziness) of hair swatches treated with composition 1A according to an embodiment of the disclosure and comparative compositions C1, C2, and C3, and a control hair swatch treated with water, after 24 hours of exposure to a condition of 80% relative humidity (80% RH) at 27° C.

FIG. 3 is a set of RUMBA 2D images of hair swatches treated with composition 1 A according to an embodiment of the disclosure and comparative compositions C1, C2, and C3, and a control hair swatch treated with water, after 24 hours of exposure to a condition of 80% relative humidity (80% RH) at 27° C.

FIG. 4 is a graph showing the denaturation temperatures of hair swatches treated with composition 1A according to an embodiment of the disclosure and comparative compositions C1, C2, and C3, and a control hair swatch treated with water.

FIG. 5 is a graph showing the cycles to break (CTB) of hair swatches treated with composition 1A according to an embodiment of the disclosure and comparative compositions C1, C2, and C3, and a control hair swatch treated with water.

FIG. 6 is a graph showing the performance of composition 1A according to an embodiment of the disclosure and compositions C1 and C2, and a control composition (water) on repairing split ends of hair evaluated on curly hair swatches, compared to an untreated control curly hair swatch.

FIG. 7 is a set of photographs of two half-head studies showing hair treated with composition 1 A (left side) compared to hair treated with deionized water (right side).

FIG. 8 is a graph illustrating the denaturation temperatures of unbleached (natural) curly hair swatches and pre-bleached curly hair swatches when the hair was untreated (control) or treated with composition 1A once (1×), three times (3×), and five times (5×).

FIGS. 9A-9B are RUMBA 2D images of hair swatches treated with compositions according to the disclosure, swatches treated with comparative compositions, and a control swatch, obtained during the course of a lastingness study.

FIGS. 10A-10C are photographs of swatches treated with compositions according to the disclosure, swatches treated with comparative compositions, and control swatches, obtained during the course of a lastingness study.

FIGS. 11A-11B are graphical representations comparing the frizz control (FIG. 11A) and curl definition (FIG. 11B) of swatches treated with compositions according to the disclosure with swatches treated with comparative compositions and control swatches during a lastingness study.

It is to be understood that the foregoing and following descriptions are exemplary and explanatory only and are not intended to be restrictive of any subject matter claimed.

### DETAILED DESCRIPTION

The present disclosure relates to compositions for treating keratin fibers, such as treating curly or wavy hair for maintaining and/or improving curl definition and hold, preventing unwanted curl elongation, providing shape control and discipline, providing frizz control and volume control, improving hair integrity, and/or preventing and/or reducing hair damage. The present disclosure also relates to methods of using the compositions disclosed herein for providing overall benefits to hair, particularly curly or wavy hair.

The disclosure relates to compositions comprising (a) at least one carboxylic acid or salt thereof; (b) at least one cyclodextrin or derivative thereof; (c) at least one polysaccharide thickening agent; (d) at least one polyol; and (c) water. The pH of the composition is generally less than 7. The compositions can provide lasting curl definition, frizz and/or volume control, and/or hair integrity, as well as additional sensorial benefits to hair, even in high humidity. The disclosure also relates to methods of making and using the compositions on hair in styling routines to maintain curl definition, reduce frizz, improve durability, improve hair integrity, and enhancing sensorial properties of the hair, particularly curly and wavy hair.

I. Compositions

In various embodiments, compositions according to the disclosure comprise (a) at least one carboxylic acid or salt thereof; (b) at least one cyclodextrin or derivative thereof; (c) at least one polysaccharide thickening agent; (d) at least one polyol; and (e) water. The pH of the composition is generally less than 7.

### Carboxylic Acids or Salts Thereof

Compositions according to the disclosure comprise at least one carboxylic acid or salt thereof. Mixtures of carboxylic acids and/or salts of carboxylic acids may be chosen. As used herein, the term “carboxylic acid” includes salts of carboxylic acids whether or not expressly stated. The carboxylic acids which may be used include, in various embodiments, mono-carboxylic acids (one carboxylic group) or poly-carboxylic acids (two or more carboxylic groups). Preferably, the carboxylic acids are non-polymeric carboxylic acids. The carboxylic acids may be chosen, in certain embodiments, from those having a molecular weight of less than about 900 g/mol, such as less than about 700 g/mol, less than about 500 g/mol, less than about 300 g/mol, or less than about 200 g/mol.

In some embodiments, the at least one carboxylic acid may be chosen from mono-carboxylic acids such as formic acid, acetic acid, propionic acid, butyric acid, valeric acid, caproic acid, entanthic acid, caprylic acid, pelargonic acid, capric acid, undecylic acid, lauric acid, tridecylic acid, myristic acid, pentadecylic acid, palmitic acid, margaric acid, stearic acid, nonadecylic acid, arachidic acid, lactic acid, or salts thereof.

In further embodiments, the at least one carboxylic acid may be chosen from di-carboxylic acids such as oxalic acid, malonic acid, malic acid, glutaric acid, citraconic acid, succinic acid, adipic acid, tartaric acid, fumaric acid, maleic acid, sebacic acid, azelaic acid, dodecanedioic acid, phthalic acid, isophthalic acid, terephthalic acid, 2,6-naphthalene dicarboxylic acid, or salts thereof.

In yet further embodiments, the at least one carboxylic acid may be chosen from tri-carboxylic acids such as citric acid, isocitric acid, aconitric acid, propane-1,2,3-tricarboxylic acid, benzene-1,3,5-tricarboxylic acid, or salts thereof.

In still further embodiments, the at least one carboxylic acid may be chosen from poly-carboxylic acids that have more than three carboxylic groups, such as phytic acid.

In various embodiments, the compositions comprise a mixture of two or more carboxylic acids and/or salts thereof. For example, the compositions may comprise a mixture of two or more mono-carboxylic acids and/or salts thereof, or may comprise a mixture or two or more poly-carboxylic acids and/or salts thereof, such as two or more di-carboxylic acids, two or more tri-carboxylic acids, or a mixture of one or more di-carboxylic acids and one or more tri-carboxylic acids, and so on. Mixtures of one or more mono-carboxylic or salts thereof with one or more poly-carboxylic acids or salts thereof are also contemplated. In further embodiments, the composition comprises only one carboxylic acid or a salt thereof.

In certain embodiments, the at least one carboxylic acid is chosen from citric acid, lactic acid, malic acid, maleic acid, tartaric acid, glycolic acid, phytic acid, salts thereof, or a mixture of two or more of the foregoing.

In various embodiments, the compositions comprise a total amount of carboxylic acids and/or salts thereof ranging from about 0.01% to about 20% by weight, such as about 0.1% to about 18%, about 0.5% to about 15%, or about 1% to about 13% by weight, relative to the total weight of the composition. By way of non-limiting example, the compositions may comprise a total amount of carboxylic acids and/or salt thereof ranging from about 0.5% to about 15%, by weight, for example, from about 0.5% to about 12%, from about 0.5% to about 10%, from about 0.5% to about 9%, from about 0.5% to about 8%, from about 0.5% to about 7%, from about 0.5% to about 6%, from about 0.5% to about 5%, from about 0.5% to about 4%, from about 0.5% to about 3%, from about 1% to about 15%, from about 1% to about 12%, from about 1% to about 10%, from about 1% to about 9%, from about 1% to about 8%, from about 1% to about 7%, from about 1% to about 6%, from about 1% to about 5%, from about 1% to about 4%, from about 1% to about 3%, from about 2% to about 15%, from about 2% to about 12%, from about 2% to about 10%, from about 2% to about 9%, from about 2% to about 8%, from about 2% to about 7%, from about 2% to about 6%, or from about 2% to about 5%, by weight, relative to the total weight of the composition, including all ranges and subranges thereof. In some embodiments, the compositions comprise a total amount of carboxylic acids and/or salts thereof ranging from about 0.5% to about 13%, such as from about 0.5% to about 10%, or from about 0.5% to about 8% by weight, relative to the total weight of the composition.

In some embodiments, the at least one carboxylic acid comprises or consists of citric acid, lactic acid, malic acid, maleic acid, tartaric acid, glycolic acid, phytic acid, salts thereof, or a mixture thereof, in an amount ranging from about 0.1% to about 10%, such as from about 0.3% to about 9%, or from about 0.5% to about 8% by weight, relative to the total weight of the composition, including all ranges and subranges thereof.

In still further embodiments, the composition comprises citric acid and/or salts thereof in an amount ranging from about 1% to about 5%, such as from about 1% to about 4%, from about 1% to about 3%, from about 1.5% to about 4%, from about 1.5% to about 3.5%, from about 1.5% to about 3%, from about 2% to about 4%, from about 2% to about 3.5%, from about 2% to about 3%, or from about 2.25% to about 2.75%, including all ranges and subranges thereof, or is about 1%, about 1.5%, about 2%, about 2.5%, about 3%, or about 3.5% by weight, relative to the total weight of the composition, including all ranges and subranges using any of the foregoing as upper or lower limits.

### Cyclodextrins or Derivatives Thereof

Compositions according to the disclosure comprise at least one cyclodextrin or derivative thereof. As used herein, the term “cyclodextrins” includes derivatives, whether or not expressly stated. Cyclodextrins are a family of cyclic oligosaccharides consisting of a macrocyclic ring of glucose subunits joined by α-1,4 glycosidic bonds.

The cyclodextrins that can be used include those of the formula:

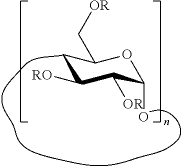

wherein:
   R is chosen from H, CH₃, or a hydroxypropyl group, and
   n ranges from 6-8.

For example, in embodiments where R=H, the cyclodextrin may be α-cyclodextrin (n=6), β-cyclodextrin (n=7), or γ-cyclodextrin (n=8). By way of example, α-cyclodextrin sold by the company WACKER under the name CAVAMAX W6 PHARMA, β-cyclodextrin sold by the company WACKER under the name CAVAMAX W7 PHARMA, or γ-cyclodextrin sold by the company WACKER under the name CAVAMAX W8 PHARMA can be used.

In other embodiments where R=CH₃, the cyclodextrin may be a methyl-cyclodextrin, such as methyl-α-cyclodextrin (n=6), methyl-β-cyclodextrin (n=7), or methyl-γ-cyclodextrin (n=8). For example, the methyl-β-cyclodextrin sold by the company WACKER under the name CAVASOL W7 may be chosen.

In various embodiments, the at least one cyclodextrin may comprise a mixture of cyclodextrins and/or derivatives thereof. For example, the at least one cyclodexctrin may be a mixture of α-cyclodextrin, β-cyclodextrin, and/or γ-cyclodextrin. In another embodiment, the at least one cyclodextrin includes β-cyclodextrin. In yet a further embodiment, the cyclodextrin is only β-cyclodextrin, and no other cyclodextrins or derivatives thereof are present in the composition.

In various embodiments, the compositions comprise a total amount of cyclodextrins and/or derivatives thereof in an amount ranging from about 0.01% to about 20% by weight, including all ranges and subranges thereof, such as from about 0.1% to about 18%, from about 0.5% to about 18%, from about 0.5% to about 15%, from about 0.5% to about 12%, from about 0.5% to about 10%, from about 0.5% to about 9%, from about 0.5% to about 8%, from about 0.5% to about 7%, from about 0.5% to about 6%, from about 0.5% to about 5%, from about 0.5% to about 4%, from about 0.5% to about 3%, from about 1% to about 18%, from about 1% to about 15%, from about 1% to about 12%, from about 1% to about 10%, from about 1% to about 9%, from about 1% to about 8%, from about 1% to about 7%, from about 1% to about 6%, from about 1% to about 5%, from about 1% to about 4%, or from about 1% to about 3% by weight, relative to the total weight of the composition. In some embodiments, the compositions comprise a total amount of cyclodextrins and/or derivatives thereof ranging from about 1% to about 20%, such as from about 0.1% to about 18%, from about 0.5% to about 15%, from about 0.5% to about 10%, from about 0.5% to about 8%, from about 1% to about 13%, from about 1% to about 7%, or from about 1% to about 5% by weight, relative to the total weight of the composition.

In some embodiments, the compositions comprises β-cyclodextrin in an amount ranging from about 0.1% to about 10%, such as from 0.2% to about 8%, from about 0.3% to about 7%, from about 0.4% to about 6%, from about 1% to about 10%, from about 1% to about 8%, from about 1% to about 5%, from about 1% to about 3% by weight, relative to the total weight of the composition, including all ranges and subranges thereof.

In various embodiments, it is particularly advantageous to choose total amounts of carboxylic acids (including salts) and cyclodextrins (including derivatives) such that a mole ratio of carboxylic acid(s):cyclodextrin(s) ranges from about 0.5:1 to about 25:3, for example about 4:1 to about 15:1, about 5:1 to about 12:1, or about 6:1 to about 10:1, such as, for example, about 1:1, about 2:1, about 3:1, about 4:1, about 5:1, about 6:1, about 7:1, about 8:1, about 9:1, about 10:1, about 11:1, about 12:1, about 13:1, about 14:1, about 15:1, about 16:1, about 17:1, about 18:1, about 19:1, about 20:1, about 3:2, about 5:2, about 7:2, about 9:2, about 11:2, about 13:2, about 15:2, about 17:2, about 19:2, about 1:3, about 2:3, about 4:3, about 5:1, about 7:3, about 8:3, about 10:3, about 11:3, about 13:3, about 14:3, about 16:3, about 17:3, about 19:3, or about 20:3, including all ranges and subranges using any of the foregoing as upper or lower limits.

In some embodiments, compositions according to the disclosure may have a weight ratio of (a) the total amount of carboxylic acids and salts thereof to (b) the total amount of cyclodextrins and derivatives thereof ranging from about 1:3 to about 6:1, for example from about 1:3 to about 3:1, from about 1:2.5 to about 2.5:1, from about 1:2 to about 2:1, from about 1:1 to about 1.75:1, from about 1.5:3 to about 3:1.5, from about 1.5:2.5 to about 2.5:1.5, or from about 1.5:2 to about 2:1.5, or may be about 1:3, about 1:2.5, about 1:2, about 1:1.5, about 1:1, about 1.5:1, about 2:1, about 2.5:1, about 3:1, about 3.5:1, about 4:1, about 4.5:1, about 5:1, about 5.5:1, about 6:1, about 1.5:3, about 1.5:2.5, about 1.5:2, about 2:1.1, about 2.5:1.5, about 3:1.5, about 3.5:1.5, about 4:1.5, about 4.5:1.5, about 5:1.5, about 5.5:1.5, or about 6:1.5, including all ranges and subranges using any of the foregoing as upper or lower limits.

### Polysaccharide Thickening Agents

Compositions according to the disclosure comprise at least one polysaccharide as a thickening agent. The term “thickening agent” may be referred to interchangeably to as “thickener” or “viscosity modifying agent.” The at least one polysaccharide agent may be chosen from anionic polysaccharide thickening agents, nonionic polysaccharide thickening agents, or mixtures of two or more thereof. Polysaccharide thickening agents that can be used include, by way of example only, inulins, celluloses, starches, guar gums, xanthan gums, pullulan gums, alginate gums, agar-agar gums, carrageenan gums, gellan gums, gum arabics, xyloses, tragacanth gums, or derivatives of the foregoing. In further embodiments, cellobiose, maltodextrin, scleroglucan, chitosan, ulvan, fucoidan, alginate, pectin, or heparin. In some embodiments, mixtures of two or more of any of these may also be chosen.

Non-limiting examples of gums include acacia, agar, algin, alginic acid, ammonium alginate, amylopectin, calcium alginate, calcium carrageenan, carnitine, carrageenan, dextrin, gelatin, gellan gum, guar gum, hectorite, hyaluronic acid, hydrated silica, hydroxypropyl chitosan, hydroxypropyl guar, karaya gum, kelp, locust bean gum, natto gum, potassium alginate, potassium carrageenan, propylene glycol alginate, sclerotium gum, sodium carboxymethyl dextran, sodium carrageenan, tragacanth gum, xanthan gum, dehydroxanthan gum, and biosacharide gum. Modified gums or derivatives of gums may also be used, such as, for example, deacylated gellan gum, welan gum, or hydroxypropylated guar gum, such as Jaguar HP 105 sold by Rhodia.

In some embodiments, suitable polysaccharide thickening agents that can be used are chosen from gums, such as gum arabic, tragacanth gum, karaya gum, guar gum, gellan gum, tara gum, locust bean gum, tamarind gum, xanthan gum, dehydroxanthan gum, Seneca gum, sclerotium gum, gellan gum, and the like.

Non-limiting examples of celluloses include hydroxyalkylcelluloses, such as hydroxymethylcellulose, methylhydroxyethylcellulose, hydroxyethylcelluloses, hydroxypropylmethylcellulose, hydroxybutylcellulose, hydroxyethylmethylcellulose (also known as methyl hydroxyethylcellulose) or hydropropylcelluloses, which may or may not contain a fatty chain. One particularly suitable hydroxypropylmethylcellulose is Methocel F4M sold by Dow Chemicals (INCI name: hydroxypropylmethylcellulose). Celluloses modified with groups comprising one or more nonionic fatty chains that can be used include hydroxyethylcelluloses, preferably nonionic hydroxyethylcelluloses, modified by groups comprising at least one fatty chain, such as alkyl, arylalkyl or alkylaryl groups, or their mixtures, and in which the alkyl groups are preferably C8-C22 alkyl groups, such as the product NATROSOL™ Plus Grade 330 CS (C16 alkyls), sold by Aqualon, corresponding to the INCI name cetylhydroxyethylcellulose, or the product BERMOCOLL® EHM 100 sold by Berol Nobel, and those modified with alkylphenyl polyalkylene glycol ether groups, such as the product AMERCELL POLYMER® HM-1500 (nonylphenyl polyethylene glycol (15) ether) sold by Amerchol that corresponds to the INCI name nonoxynyl hydroxyethylcellulose. Other particular thickening agents suitable for use in the compositions described herein are hydroxypropyl guar gums.

Non-limiting examples of starches include modified starches, starch-based polymers, methylhydroxypropyl starch, potato starch, modified potato starch, wheat starch, rice starch, starch crosslinked with octenyl succinic anhydride, starch oxide, dialdehyde starch, dextrin, British gum, acetyl starch, starch phosphate, carboxymethyl starch, hydroxyethyl starch, and hydroxypropyl starch.

In some embodiments, the polysaccharide thickening agents are chosen from carrageenan, xanthan gum, cellulose, carboxymethylcellulose, hydroxyethylcellulose, hydroxypropylcellulose, hydroxypropylmethylcellulose, methylcellulose, starch, starch acetate, hydroxyethyl starch, hydroxypropyl starch, guar gum, carboxymethyl guar gum, carboxymethylhydroxypropylguar gum, hydroxyethyl guar gum, hydroxypropyl guar, xylose gum, cellobiose, dextrin, maltodextrin, scleroglucan, chitosan, ulvan, fucoidan, alginate, pectin, heparin, inulin, levan, or mixtures thereof. In further embodiments, the polysaccharide thickening agents are chosen from sclerotium gum, carrageenan, xanthan gum, cellulose gum, cyclodextrin, or mixtures of two or more thereof.

In various exemplary embodiments, the total amount of polysaccharide thickening agents may vary, and may typically range from about 0.05% to about 5%, such as from about 0.1% to about 5%, from about 0.1% to about 4%, from about 0.1% to about 3%, from about 0.1% to about 2%, from about 0.1% to about 1%, from about 0.5% to about 5%, from about 0.5% to about 4%, from about 0.5% to about 3%, from about 0.5% to about 2%, from about 0.5% to about 1%, from about 1% to about 5%, from about 1% to about 4%, from about 1% to about 3%, or from about 1% to about 2% by weight, relative to the total weight of the composition, including all ranges and subranges thereof. In some embodiments, the total amount of polysaccharide thickening agents may range from about 0.1% to about 5%, such as from about 0.25% to about 4%, from about 0.5% to about 3%, or from about 0.5% to about 2.5%, or may be about 1%, about 1.5%, about 2%, about 2.5%, about 3%, or about 3.5% by weight, relative to the total weight of the composition, including all ranges and subranges using any of the foregoing as upper or lower limits.

For example, the compositions may, in exemplary embodiments, comprise a total amount of polysaccharide thickening agents ranging from about 0.1% to about 5%, from about 0.5% to about 4%, or from about 0.75% to about 3%, preferably from about 0.5% to about 2% by weight, relative to the total weight of the composition, wherein the polysaccharide thickening agent comprises at least one polysaccharide thickening agent may be chosen from gum arabic, tragacanth gum, karaya gum, guar gum, gellan gum, tara gum, locust bean gum, tamarind gum, xanthan gum, dehydroxanthan gum, Seneca gum, sclerotium gum, gellan gum, etc. In various embodiments, the composition comprises xanthan gum in an amount ranging from about 0.1% to about 5%, such as from about 0.25% to about 4%, from about 0.5% to about 3%, or from about 0.5% to about 2.5%, or may be about 1%, about 1.5%, about 2%, about 2.5%, about 3%, or about 3.5% by weight, relative to the total weight of the composition, including all ranges and subranges using any of the foregoing as upper or lower limits.

### Polyols

Compositions according to the disclosure comprise at least one polyol. By way of example only, the at least one polyol may be chosen from C₂-C₁₆ polyols, such as C₂-C₁₂ or C₂-C₈ polyols. The at least one polyol may optionally be chosen from diols and triols. The at least one polyol may be linear or branched, saturated or unsaturated, and substituted or unsubstituted. Any stereoisomer of the polyols may also be used. Polyol(s) in the combination disclosed herein may act as conditioning or smoothing agents and improve overall sensorial properties of hair and enhance curl definition.

In various exemplary embodiments, the polyols may be chosen from glycols such as ethylene glycol, propylene glycol, butylene glycol, hexylene glycol, pentylene glycol, 1,3-propanediol, diethylene glycol, triethylene glycol, tetraethylene glycol, pentaethylene glycol, dipropylene glycol, caprylyl glycol, glycerin, diglycerin, ethylhexyglycerin, or mixtures of two or more thereof.

In other embodiments, the polyols may be chosen from, for example, propanediol, butanediol, pentanediol, hexanediol, heptanediol, octanediol, 1,2,6-hexanetriol, 1,2,4-butanetriol, trimethylolpropane, 2-butene-1,4-diol, 2-ethyl-1,3-hexanediol, 2-methyl-2,4-pentanediol, caprylyl glycol, 1,2-hexanediol, 1,2-pentanediol, 2-ethyl-2-methyl-1,3-propanediol, 3,3-dimethyl-1,2-butanediol, 2,2-diethyl-1,3-propanediol, 2-methyl-2-propyl-1,3-propanediol, 2,4-dimethyl-2,4-pentanediol, 2,5-dimethyl-2,5-hexanediol, 5-hexene-1,2-diol, 2-ethyl-1,3-hexanediol, 4-methyl-1,2-pentanediol, or mixtures of two or more thereof.

In some embodiments, the compositions according to the disclosure comprise at least two polyols, for example at least two polyols chosen from diols and/or triols. As one non-limiting example, a composition according to the disclosure may comprise a combination of at least one glycerin compound and at least one additional glycol compound.

In various embodiments, the total amount of polyols can be about 1% or more, such as about 2% or more, about 3% or more, about 4% or more, about 5% or more, about 6% or more, for example up to about 10% by weight, based on the total weight of the composition. In some embodiments, the total amount of polyol is present in the composition with a total amount ranging from about 0.1% to about 10%, including all subranges therebetween, such as from about 0.1% to about 9%, from about 0.1% to about 8%, from about 0.1% to about 7%, from about 0.1% to about 6%, from about 0.1% to about 5%, from about 0.1% to about 4%, from about 0.1% to about 3%, from about 0.1% to about 2%, from about 0.1% to about 1%, from about 0.5% to about 10%, from about 0.5% to about 8%, from about 0.5% to about 7%, from about 0.5% to about 6%, from about 0.5% to about 5%, from about 0.5% to about 4% by weight, from about 0.5% to about 3%, from about 0.5% to about 2%, or from about 0.5% to about 1.5% by weight, relative to the total weight of the composition. In some embodiments, the total amount of polyols ranges from about 0.1% to about 5%, such as from about 0.25% to about 4%, from about 0.5% to about 3%, or from about 0.5% to about 2.5%, or may be about 1%, about 1.5%, about 2%, about 2.5%, about 3%, or about 3.5% by weight, relative to the total weight of the composition, including all ranges and subranges using any of the foregoing as upper or lower limits.

In some exemplary and non-limiting embodiments, the compositions comprise at least one polyol chosen from diols and/or triols, and the total amount of polyols is about 1% or more, about 1.5% or more, about 2% or more, about 3% or more, about 4% or more, or about 5% or more by weight, for example up to about 10% by weight, relative to the total weight of the composition, and preferably comprises glycerin.

### Water

Compositions according to the disclosure comprise water. In various embodiments, the amount of water in a composition disclosed herein is generally greater than 50%, and may, for example, range from about 70% to about 98%, including all subranges therebetween, such as from about 75% to about 98%, from about 80% to about 98%, from about 85% to about 98%, from about 90% to about 98%, from about 95% to about 98%, from about 70% to about 95%, from about 75% to about 95%, from about 80% to 95%, from about 85% to 95% by weight, relative to the total weight of the composition.

### Additional Components

Compositions according to the disclosure may optionally comprise one or more additional components suitable for use in such compositions. Non-limiting examples of such additional components are provided below.

### pH Adjusters

Compositions disclosed herein may include one or more pH adjusters to increase or decrease the overall pH of the composition. For example, one or more acids may be included to decrease the pH of the cosmetic composition. Examples of suitable acids for decreasing the pH of the compositions may include one or more acids. The cosmetic composition may include one or more bases, such as sodium hydroxide, potassium hydroxide and the like, to increase the pH of the cosmetic composition. Additional or alternative acids and bases that are suitable for adjusting the pH of the compositions are readily known to one of ordinary skill in the art. For instance, in one embodiment, the pH adjuster is sodium hydroxide solution such as 0.1 M NaOH.

The amount of the pH adjuster in the compositions may be based on the desired pH of the final composition and/or product for improving curl definition and curl regularity, and/or preventing curl elongation. For example, the total amount of the pH adjuster may range from about 0.0001% to about 10% by weight, including ranges and sub-ranges therebetween, based on the total weight of the cosmetic composition.

### Preservatives

One or more preservatives may be included in the compositions described herein for treating hair. Suitable preservatives include, but are not limited to, glycerin containing compounds (e.g., glycerin or ethylhexylglycerin or phenoxyethanol), benzyl alcohol, parabens (methylparaben, ethylparaben, propylparaben, butylparaben, isobutylparaben, etc.), sodium benzoate, benzoic acid, chlorhexidine digluconate, ethylenediamine-tetraacetic acid (EDTA), potassium sorbate, and/or grapefruit seed extract, or a mixture thereof. Other preservatives are known in the cosmetics industries and include salicylic acid, DMDM Hydantoin, Formaldahyde, Chlorphenism, Triclosan, Imidazolidinyl Urea, Diazolidinyl Urea, Sorbic Acid, Methylisothiazolinone, Sodium Dehydroacetate, Dehydroacetic Acid, Quaternium-15, Stearalkonium Chloride, Zinc Pyrithione, Sodium Metabisulfite, 2-Bromo-2-Nitropropane, Chlorhexidine Digluconate, Polyaminopropyl biguanide, Benzalkonium Chloride, Sodium Sulfite, Sodium Salicylate, Citric Acid, Neem Oil, Essential Oils (various), Lactic Acid, Vitamin E (tocopherol), or mixtures thereof. In some cases, the hair-treatment compositions may include one or more preservatives selected from the group consisting of sodium benzoate, benzoic acid, chlorhexidine digluconate, chlorhexidine dihydrochloride, salicylic acid, phenoxyethanol, methyl paraben, or mixtures thereof.

The total amount of the one or more preservatives, when present, may vary. In some cases, the total amount of the one or more preservatives may range from about 0.01% to about 5%, from about 0.01% to about 4%, from about 0.01% to about 2%, from about or about 0.01% to about 3% by weight, relative to the total weight of the composition.

### Auxiliary Components

Compositions according to the disclosure may optionally comprise any auxiliary component suitable for use in such compositions. Such components may include, but are not limited to, dyes/pigments for adding color to the composition, moisturizing agents, fatty substances, thickeners other than those previously described, fillers, structuring agents, shine agents, antioxidants or penetrants, sequestrants, fragrances, buffers, dispersants, plant extracts, such as apricot seed powder, opacifiers, sunscreen agents, vitamins, and antistatic agents. Compositions according to the disclosure preferably do not include dyes/pigments for coloring the hair.

Optional auxiliary components may be present in an amount ranging up to about 15%, such as from about 0.001% to about 10%, from about 0.01% to about 5%, or from about 0.01% to about 1% by weight, relative to the total weight of the composition.

In various embodiments, the compositions are acidic and have a pH of less than 7. For example, the pH of the composition may range from about 1.5 to about 6.8, from about 2 to about 6.5, from about 2.5 to about 6, from about 3 to about 6.5, from about 3 to about 6, from about 3 to about 5.5, from about 3 to about 5, from about 3 to about 4, from about 3.5 to about 6.5, from about 3.5 to about 6, from about 3.5 to about 5.5, from about 3.5 to about 5, from about 3.5 to about 4.5, or from about 3.5 to about 4, including all ranges and subranges using any of the foregoing as upper or lower limits. In some exemplary embodiments, the pH of the composition ranges from about 3 to about 6.5. In further exemplary embodiments, the pH of the composition ranges from about 3 to about 4.

Compositions according to the disclosure may be rinse-off compositions or leave-in compositions, and are typically in the form of a gel, liquid, cream, or lotion, but are not so limited. In some embodiments, the compositions are a non-aerosol type product, which may be dispensed from a bottle, tube, or spray-bottle.

In some embodiments, the compositions may be a hair treatment product, a hair styling product, or a hair styling and managing product that can be used for styling hair while providing treatment and/or conditioning, as well as manageability benefits to the hair.

### Pre-Phase Mixture

The disclosure also relates to pre-phase mixtures, or stock mixtures, of concentrated combinations of (a) at least one carboxylic acid or salt thereof; (b) at least one cyclodextrin or derivative thereof; (c) at least one polysaccharide thickening agent; and (d) at least one polyol. For example, the pre-phase mixtures can be produced by combining and mixing particular carboxylic acid(s), cyclodextrin(s), polysaccharide thickening agent(s), polyol(s) as desired herein and particular amounts thereof with specific mole and/or ratios as described herein. The pre-phase mixture can subsequently be diluted, e.g. with water, or added into a base composition including additional components disclosed herein to produce the compositions according to the present disclosure before the composition is applied to the hair.

In various embodiments, in the pre-phase mixture, it may be particularly advantageous to choose total amounts of carboxylic acids (including salts) and cyclodextrins (including derivatives) so that a mole ratio of carboxylic acid(s):cyclodextrin(s) ranges from about 0.5:1 to about 25:3, preferably from about 4:1 to about 15:1, from about 5:1 to about 12:1, or from about 6:1 to about 10:1, or may be present in mole ratios of about 1:1, about 2:1, about 3:1, about 4:1, about 5:1, about 6:1, about 7:1, about 8:1, about 9:1, about 10:1, about 11:1, about 12:1, about 13:1, about 14:1, about 15:1, about 16:1, about 17:1, about 18:1, about 19:1, about 20:1, about 3:2, about 5:2, about 7:2, about 9:2, about 11:2, about 13:2, about 15:2, about 17:2, about 19:2, about 1:3, about 2:3, about 4:3, about 5:1, about 7:3, about 8:3, about 10:3, about 11:3, about 13:3, about 14:3, about 16:3, about 17:3, about 19:3, or about 20:3, including all ranges and subranges using any of the foregoing as upper or lower limits.

Correspondingly, in the pre-phase mixture, it may be particularly advantageous to choose total amounts of carboxylic acids (including salts) and cyclodextrins (including derivatives) so that a weight ratio of (a) the total amount of carboxylic acids and salts thereof to (b) the total amount of cyclodextrins and derivatives thereof ranges from about 1:3 to about 6:1, or may be present in weight ratios of about 1:3, about 1:2.5, about 1:2, about 1:1.5, about 1:1, about 1.5:1, about 2:1, about 2.5:1, about 3:1, about 3.5:1, about 4:1, about 4.5:1, about 5:1, about 5.5:1, or about 6:1, including all ranges and subranges using any of the foregoing as upper or lower limits.

II. Methods

### Methods of Making the Compositions

The disclosure also relates to methods of making compositions described herein. It may, in some embodiments, be particularly beneficial to choose specific mole or weight ratios of carboxylic acid(s) and/or salt(s), cyclodextrin(s) and/or derivative(s) thereof, and/or solvent(s) (e.g. deionized (DI) water). For example, by choosing specific mole or weight ratios of these components, it is possible to tailor the compositions so that greater or lesser degrees of curl definition and/or frizz control may be imparted.

Therefore, it may be useful to prepare a pre-mixture, or stock mixture, of concentrated combinations of carboxylic acid(s) and/or salt(s) thereof, cyclodextrin(s) and/or derivative(s) thereof, and solvent(s) with particular mole ratios or weight ratios thereof. In one embodiment, the pre-mixture comprises at least one carboxylic acid chosen from citric acid, and at least one cyclodextrin chosen from β-cyclodextrin.

In various embodiments, in the pre-mixture, carboxylic acids (including salts) and cyclodextrins (including derivatives) may be present in a mole ratio of carboxylic acid(s):cyclodextrin(s) ranging from about 0.5:1 to about 25:3, such as from about 4:1 to about 15:1, from about 5:1 to about 12:1, or from about 6:1 to about 10:1, or may be present in mole ratios of about 1:1, about 2:1, about 3:1, about 4:1, about 5:1, about 6:1, about 7:1, about 8:1, about 9:1, about 10:1, about 11:1, about 12:1, about 13:1, about 14:1, about 15:1, about 16:1, about 17:1, about 18:1, about 19:1, about 20:1, about 3:2, about 5:2, about 7:2, about 9:2, about 11:2, about 13:2, about 15:2, about 17:2, about 19:2, about 1:3, about 2:3, about 4:3, about 5:1, about 7:3, about 8:3, about 10:3, about 11:3, about 13:3, about 14:3, about 16:3, about 17:3, about 19:3, or about 20:3, including all ranges and subranges using any of the foregoing as upper or lower limits. Correspondingly, the weight ratio of (a) the total amount of carboxylic acids and salts thereof to (b) the total amount of cyclodextrins and derivatives thereof ranging from about 1:3 to about 6:1, or may be present in weight ratios of about 1:3, about 1:2.5, about 1:2, about 1:1.5, about 1:1, about 1.5:1, about 2:1, about 2.5:1, about 3:1, about 3.5:1, about 4:1, about 4.5:1, about 5:1, about 5.5:1, or about 6:1, including all ranges and subranges using any of the foregoing as upper or lower limits.

Once the particular carboxylic acid(s), cyclodextrin(s), and solvent(s), as well as the desired mole and/or weight ratios thereof, are chosen, the pre-mixture may be prepared by any conventional method. As an illustratory example only, the pre-mixture may be prepared by simple mixing of the components at the chosen ratio, optionally with heating. For example, the mixture may be mixed at a temperature ranging from about 40° C. to about 100° C., such as about 50° C. to about 95° C. By way of example, the pre-mixture may be prepared by: first, mixing one or more carboxylic acids and/or salts thereof and one or more cyclodextrins and/or derivatives thereof in DI water; second, heating the mixture in a water bath at about 80° C. until the contents dissolve and the mixture forms a viscous liquid; third, leaving the mixture at about 80° C. for about 2 hours to allow the ingredients to complete the reaction, and; fourth, cooling the mixture to room temperature.

After the pre-mixture is prepared, it may then be diluted with additional solvent and other additives including polysaccharide(s) and polyol(s), e.g., xanthan gum and glycerin, to the desired concentration of the final composition according to the disclosure. The additional solvent may be the same as the solvent in the pre-mixture, may be different from the solvent in the pre-mixture, or may be a combination of the solvent in the pre-mixture with one or more solvents other than that in the pre-mixture.

In one exemplary and non-limiting embodiment, a composition according to the present disclosure comprises citric acid, beta-cyclodextrin, xanthan gum, and glycerin, may be prepared by diluting a pre-mixture of citric acid and beta-cyclodextrin to a desired concentration with additional solvent(s), e.g., DI water, adjusting the pH of the dilution with a pH adjuster, e.g., 0.1 M sodium hydroxide solution, subsequentially adding desired amounts of xanthan gum and glycerin to the dilution to obtain a mixture, and further mixing the mixture using a mixer or vortexer (or vortex mixer) to arrive at the compositions disclosed herein.

In various embodiments, compositions according to the disclosure may contain a weight ratio of pre-mixture:additional components ranging from about 1:99 to about 50:50. For example, compositions according to the disclosure may contain a weight ratio of pre-mixture:additional components ranging from about 2:98 to about 40:60 or about 3:97 to about 35:65, for example may be about 2.5:97.5, about 5:95, about 7.5:92.5, about 10:90, about 12.5:87.5, about 15:85, about 17.5:81.5, about 20:80, about 25:75, or about 30:70, including all ranges and subranges using any of the foregoing as upper or lower limits. In some exemplary embodiments, the weight ratio of pre-mixture:additional components ranges from about 5:95 to about 15:85.

### Methods of Treating Hair

The disclosure also relates to methods of treating keratin fibers, particularly curly or wavy hair using the compositions disclosed herein, for example to improve curl definition and/or curl durability, improve frizz and/or volume control, and/or maintain and/or enhance integrity of hair in a styling routine. The methods generally comprise applying any of the compositions according to the disclosure to the keratin fibers, particularly curly or wavy hair. The degree of curliness or curl type of the hair may vary and is not limited. For example, the curls of hair that may be treated may range from slightly wavy to tight curls, and may have different textures and colors. The curls of the hair may be natural or unnatural, such as obtained via a permanent hair styling process using chemicals that structurally change the texture of the hair. In some embodiments, the methods disclosed herein may include changing the shape of the hair, for example, transforming straight hair to curly hair, before, during, or after treating the hair with the compositions disclosed herein (e.g. via a permanent wave process).

In some embodiments, methods according to the disclosure may include applying an effective amount of a composition disclosed herein to hair having curls, when the hair is dry, wet, damp, or moist when the composition is applied thereto. As used herein, the term “effective amount” refers to an amount sufficient to provide a desired effect to the hair, taking into account the degree of curliness, the length, the volume, and the texture of the hair. In general, from about 0.1 grams to about 5 grams of product is applied per gram of hair, depending on the specific product formulation, hair length, hair volume, and hair style type. The composition may be applied to the hair after the hair is shampooed and/or before the hair is conditioned, or may be applied to the hair without shampooing or conditioning the hair before or after such application.

After the composition is applied on the hair, the hair may be massaged or combed through with fingers or by a means such as a comb or the like such that the composition was evenly distributed on the hair. If desired, after the hair is treated with the compositions, while the hair is still wet or damp, the hair may be styled by scrunching, twisting, or the like, which may aid in defining the curls. The hair treated with the composition may be air dried or dried by a dryer, for example a hood dryer, where the composition applied to the hair may be rinsed off before drying or allowed to remain on the hair as a leave-in product for any period of time as desired, for example, a few hours, overnight, or a few days, or until the next washing or rinsing of the hair. After the treated hair is dried, the hair may be passed by fingers to break the film formed on the hair by the ingredients in the components.

For example, in one exemplary and non-limiting treatment routine, a composition disclosed herein is applied after the hair is shampooed and rinsed. After the composition is applied to the hair and distributed substantially evenly, the hair may be styled, such as by scrunching using hands for a couple of times, and then air-dried or dried using a dryer, where the composition is allowed to remain on the hair as a leave-in composition for any period of time as desired, for example, a few hours, overnight, or a few days, or until the next washing or rinsing of the hair. After the treated hair is dried, the hair may optionally be passed by fingers to break the film formed on the hair by the ingredients in the components. In this treatment routine, the composition is used as a leave-in product.

In another exemplary and non-limiting treatment routine, a composition disclosed herein is applied optionally after the hair is shampooed and rinsed. After the composition is applied and distributed substantially evenly on the hair, the hair is further treated with a conditioner, where the conditioner and the composition are allowed to remain on the hair for a desired period of time, for example, about 1, 2, 5, 10, 20, 30, or 60 minutes or longer, and then the hair is rinsed, optionally styled (e.g. scrunched), and air-dried or dried with a dryer. In this routine, the composition is used as a rinse-off product. The conditioner that can be used include any types of conditioners, such as standard conditioners or conditioners that can provide additional benefits other than conditioning, for example, detangling benefit.

In another exemplary and non-limiting treatment routine, a composition disclosed herein is applied to the hair when the hair is dry, wet, or damp, where the hair is not pre-washed with a shampoo (e.g. separate from a hair cleansing routine). After the composition is applied to the hair it can be substantially evenly distributed on the hair, optionally allowed to remain on the hair for a desired period of time, for example about 30 second, 1, 2, 5, 10, 20, 30, or 60 minutes, or longer, and then, with or without rinsing the composition from the hair, the hair is styled and air-dried or dried with a dryer. No shampoo or conditioner is used in this routine.

Generally, when the composition is rinsed from the treated hair, the hair looks less stiff and not crunchy, as is often seen with traditional styling compositions.

In some further embodiments, methods according to the present disclosure may involve the use of heat, although heat is not required per se. For example, an exemplary method may comprise applying to hair a composition disclosed herein and heating the hair while the composition is still on the hair and/or after the composition is rinsed off. In some embodiments, the hair may be washed with a shampoo and rinsed before applying the composition to the hair. In some embodiments, the hair treated with the composition may be further treated with a conditioner. The conditioner may be applied to the hair when the composition disclosed herein is still on the hair, or after the composition disclosed herein is rinsed off. In some embodiments, the hair may be heated while the composition disclosed herein and/or the conditioner is still on the hair, or after the composition disclosed herein and/or the conditioner is rinsed off. In some embodiments, the hair may be rinsed to remove the compositions disclosed herein and/or the conditioner after heating the hair.

In various embodiments, the heating apparatus or devices that can be used to heat the hair include hair dryers such as hood dryers and blow dryers, a steamer, or a flat iron. The hair may be heated at a temperature ranging from about 50° C. to about 225° C. for a desired period of time, such as from about 1 minutes to about 5 minutes. In some embodiments, the hair may be heated using a temperature of, or with a device capable of achieving a temperature of, about 100° C. to about 225° C., about 120° C. to about 225° C., about 150° C. to about 225° C., about 180° C. to about 225° C., about 100° C. to about 200° C., about 150° C. to about 200° C. For example, a hair dryer may be used that can expel air having a temperature in the range of about 100° C. to about 225° C.

In one exemplary and non-limiting routine, a method according to the present disclosure comprises applying to hair any of the compositions disclosed herein; drying the hair using a hair dryer at a temperature of about 60° C. to about 80° C. for about 2 to 3 minutes or until the hair is approximately 80% dry; and subsequently, applying a flat iron on the hair for about 2-5 passes, each pass taking about 10 seconds, where the flat iron is set at a temperature ranging from about 180° C. to about 225° C.

In some embodiments, the heat treatment described above may change the shape of the hair. For example, the heat applied to the hair may transform straight hair to curly hair. Compositions according to the present disclosure may be applied to the hair before, during, or after the hair is heated and the shape of the hair is changed, to improve the durability of the new shapes and reduce or minimize the damage to the hair caused during heating, as well as to provide other sensorial benefits such as frizz control and smoothness to the hair.

In some further embodiments, the disclosure relates to methods of treating hair using any of the compositions disclosed herein after the hair is colored to protect the color imparted on the hair, enhance hair integrity, and/or provide frizz and/or volume control to the hair. The methods may generally include applying to the hair a treatment composition comprising (a) at least one carboxylic acid or salt thereof; (b) at least one cyclodextrin or derivative thereof; (c) at least one polysaccharide thickening agent; (d) at least one polyol; and (e) water; wherein the pH of the treatment composition is less than 7; and optionally, rinsing the treatment composition from the hair; wherein the treatment composition is applied to the hair after the hair is colored with a coloring composition comprising at least one hair colorant. In various embodiments, the at least one hair colorant may be an oxidative hair colorant, a direct dye, and/or a pigment.

In various embodiments, the methods of treating hair with the compositions according to the disclosure impart one or more of the long-lasting benefits described above to the hair, without a greasy feel or flaking, relative to hair not having been treated with a composition according to the disclosure. The term “long-lasting,” as used herein, means that the curl defining, prevention of elongation, volume control, frizz control, hair integrity effects, and/or other benefits imparted to the hair may remain a desired length of time, such as at least one or a few hours, a few days, even after the hair is rinsed or washed. For example, the benefits may last through at least one, at least two, at least three, at least four, at least five, at least six, or even more, shampooings.

III. Kits

The disclosure further relates to kits comprising a first container containing a composition according to the present disclosure and/or a concentrated pre-mixture as disclosed herein, and one or more additional containers containing one or more hair care/treatment compositions other than the compositions disclosed herein, such as, for example, a shampoo, a conditioner, a hair bleach composition, an oxidizing composition, a hair mask, or a hair styling composition.

The pre-mixture, or stock mixture, is a concentrated combination of (a) at least one carboxylic acid or salt thereof; (b) at least one cyclodextrin or derivative thereof; (c) at least one polysaccharide thickening agent; and (d) at least one polyol. The pre-mixture may be substantially free of water. For example, the pre-mixture may have less than about 20%, less than about 18%, less than about 15%, less than about 12%, less than about 10%, less than about 8%, less than about 5%, less than about 3%, less than about 2%, or less than about 1% water. The pre-mixture can then be diluted, e.g. with water, to result in a composition according to the disclosure near or at the time of use. The kit may optionally further comprise instructions instructing a user regarding appropriate mixing steps, ratios, timing, etc. when a pre-mixture is included, or instructions for using the compositions, including various treatment routines disclosed herein. The kits in various embodiments may comprise at least one container suitable for containing and/or dispensing the compositions described herein for applying to the hair. In some other embodiments, a kit may comprise a first container containing a composition according to the disclosure, and at least one additional container comprising a composition according to the disclosure or a composition not according to the disclosure, such as, for example, a shampoo, a conditioner, a hair mask, or a hair styling composition.

Having described the many embodiments of the present invention in detail, it will be apparent that modifications and variations are possible without departing from the scope of the disclosure defined in the appended claims. Furthermore, it should be appreciated that all examples in the present disclosure, while illustrating many embodiments of the disclosure, are provided as non-limiting examples and are, therefore, not to be taken as limiting the various aspects so illustrated. It is to be understood that all definitions herein are provided for the present disclosure only.

As used herein, the terms “comprising,” “having,” and “including” (or “comprise,” “have,” and “include”) are used in their open, non-limiting sense. The phrase “consisting essentially of” limits the scope of a claim to the specified materials or steps and those that do not materially affect the basic and novel characteristics of the compositions.

In this application, the use of the singular includes the plural unless specifically stated otherwise. The singular forms “a,” “an,” “the,” and “at least one” are understood to encompass the plural as well as the singular unless the context clearly dictates otherwise. The expression “one or more” means “at least one” and thus includes individual components as well as mixtures/combinations. Likewise, the term “a salt thereof” also relates to “salts thereof.” Thus, where the disclosure refers to “an element selected from the group consisting of A, B, C, D, E, F, a salt thereof, or mixtures thereof,” it indicates that that one or more of A, B, C, D, and F may be included, one or more of a salt of A, a salt of B, a salt of C, a salt of D, a salt of E, or a salt of F may be included, or a mixture of any two of A, B, C, D, E, F, a salt of A, a salt of B, a salt of C, a salt of D, a salt of E, or a salt of F may be included.

The term “and/or” should be understood to include both the conjunctive and the disjunctive. For example, “citric acid and/or salts thereof” means “citric acid and salts thereof” as well as “citric acid or salts thereof,” and expressly covers instances of either.

As used herein, the phrases “and mixtures thereof,” “and mixtures of two or more thereof,” “and a mixture thereof,” “and combinations thereof,” “and a combination thereof,” “or mixtures thereof,” “or a mixture thereof,” “or combinations thereof,” “or combinations of two or more thereof,” “or a combination thereof,” and the like are used interchangeably to denote that the listing of components immediately preceding the phrase, such as “A, B, C, D, or mixtures thereof” signify that the component(s) may be chosen from A, from B, from C, from D, from A+B, from A+B+C, from A+D, from A+C+D, etc., without limitation on the variations thereof. Thus, the components may be used individually or in any combination thereof.

For purposes of the present disclosure, it should be noted that to provide a more concise description, some of the quantitative expressions given herein are not qualified with the term “about.” It is understood that whether the term “about” is used explicitly or not, every quantity given herein is meant to refer to the actual given value, and it is also meant to refer to the approximation to such given value that would reasonably be inferred based on the ordinary skill in the art, including approximations due to the experimental and/or measurement conditions for such given value.

All ranges and amounts given herein are intended to include sub-ranges and amounts using any disclosed point as an end point, and all endpoints are intended to be included unless expressly stated otherwise. Thus, a range of “1% to 10%, such as 2% to 8%, such as 3% to 5%,” is intended to encompass ranges of “1% to 8%,” “1% to 5%,” “2% to 10%,” and so on. All numbers, amounts, ranges, etc., are intended to be modified by the term “about,” whether or not expressly stated, unless expressly stated otherwise. Similarly, a range given of “about 1% to 10%” is intended to have the term “about” modifying both the 1% and the 10% endpoints. The term “about” is used herein to indicate a difference of up to +/−10% from the stated number, such as +/−9%, +/−8%, +/−7%, +/−6%, +/−5%, +/−4%, +/−3%, +/−2%, or +/−1%. Likewise, all endpoints of ranges are understood to be individually disclosed, such that, for example, a range of 1:2 to 2:1 is understood to disclose a ratio of both 1:2 and 2:1.

As used herein, if a component is described as being present “in an amount up to” a certain amount, it is intended that such component is, in fact, present in the composition, i.e. is present in an amount greater than 0%.

All amounts given herein are relative to the amount of active material, unless otherwise indicated.

All percentages, parts and ratios herein are based upon the total weight of the compositions of the present disclosure, unless otherwise indicated.

As used herein, the terms “applying a composition onto keratin fibers” and “applying a composition onto hair” and variations of these phrases are intended to mean contacting the keratin materials including hair, with at least one of the compositions of the disclosure, in any manner. It may also mean contacting the keratin materials in an effective amount.

As used herein, the term “curly hair” refers to any hair including a curl. The curl may be natural or unnatural, i.e., formed by chemical treatment or physical treatment of the hair. The degree of curliness of the hair may vary and is not limited.

As used herein, hair with improved or enhanced curl definition may have curls with a shape that has a clean ringlet appearance rather than being frizzy, curls that appear more individualized, curls that are more closed in appearance, and/or curls that have an improved visual appearance of the hair color and/or highlights.

A “leave-in” composition or product refers to a composition such as a hair-treatment composition that is not rinsed and/or washed away with water or acceptable solvent after the application of the composition onto the keratin fiber, such as hair; instead, the composition is allowed to remain on the keratin fibers for a period of time as desired, such from 1 hour, 2 hours, 3 hours, 4 hours, up to 8 hours, overnight, or as long as needed, until next time of washing or rinsing the keratin fibers.

As used herein, the term “salts” refers to throughout the disclosure may include salts having a counterion such as an alkali metal, alkaline earth metal, or ammonium counterion. This list of counterions, however, is non-limiting. Salts also include a dissociated form of a compound, e.g. in an aqueous solution.

It is to be understood that, with regard to salts of carboxylic acids described herein, it is intended to encompass the use of a salt of a carboxylic acid as an ingredient added to a composition according to the disclosure, or to the salt of the carboxylic acid that forms when the carboxylic acid is used as an ingredient in a composition according to the disclosure.

As used herein, the term “substantially free” or “essentially free” as used herein means the specific material may be present in small amounts that do not materially affect the basic and novel characteristics of the compositions according to the disclosure. For instance, there may be less than 2% by weight of a specific material added to a composition, based on the total weight of the compositions (provided that an amount of less than 2% by weight does not materially affect the basic and novel characteristics of the compositions according to the disclosure. Similarly, the compositions may include less than 2%, less than 1.5%, less than 1%, less than 0.5%, less than 0.1%, less than 0.05%, or less than 0.01%, or none of the specified material. Furthermore, all components that are positively set forth in the instant disclosure may be negatively excluded from the claims, e.g., a claimed composition may be “free,” “essentially free” (or “substantially free”) of one or more components that are positively set forth in the instant disclosure. The term “substantially free” or “essentially free” as used herein may also mean that the specific material is not added to the composition but may still be present in a raw material that is included in the composition.

As used herein, the term “treat” (and its grammatical variations) refers to the application of the compositions of the present disclosure onto the surface of keratin materials, such as hair.

It is to be understood that treatment of hair “at” a particular temperature means that the hair is exposed to said temperature. For example, heating hair “at a temperature ranging from 100° C. to 150° C.” means that the device with which the hair is heated has a temperature in said range during the treatment.

Unless otherwise expressly stated, it is in no way intended that any method set forth herein be construed as requiring that its steps be performed in a specific order. Accordingly, where a method claim does not expressly recite an order to be followed by its steps or it is not specifically stated in the claims or descriptions that the steps are to be limited to a specific order, it is no way intended that any particular order be inferred.

### EXAMPLES

The following Examples are intended to be non-limiting and explanatory in nature only. In the Examples, amounts are expressed in percentage by weight (wt %) of active materials, relative to the total weight of the composition, unless otherwise indicated.

### Example 1—Compositions

Exemplary comparative compositions C1 and C2 and inventive composition 1 A were prepared according to the formulations set forth in Table 1, as described.

TABLE 1

| | |
|---|---|
| Inventive | Comparative |

| | 1A | C1 | C2 |
|---|---|---|---|
| XANTHAN GUM | 1 | 1 | |
| GLYCERIN | 1 | 1 | |
| CITRIC ACID | 2.5 | | 2.5 |
| β-CYCLODEXTRIN | 1.5 | | 1.5 |
| WATER | QS to 100 | QS to 100 | QS to 100 |

Composition C1 was prepared by mixing xanthan gum and glycerin in water, and adjusting the pH of the mixture to about 3.5-4 at 25° C. using 0.1 M sodium hydroxide (NaOH) solution. Composition C2 was prepared by diluting a premixture of citric acid and cyclodextrin in deionized water and adjusting the pH of the dilution to about 3.5-4 at 25° C. using 0.1 M NaOH solution. The premixture of citric acid (CA) and cyclodextrin (CD) was prepared by mixing citric acid, cyclodextrin, and deionized water (DI) in a mole ratio of CD:CA:DI at 1:10:50, corresponding to a weight ratio of CD:CA:DI at 29:48:23, and heating the mixture in a water bath at about 80° C. until CA and CD were dissolved in the DI water and the mixture formed into viscous liquid, leaving the mixture in the water bath for a period of about 2 hours, and then cooling down the mixture to room temperature (RT). Composition 1A was prepared by diluting the premixture of citric acid and cyclodextrin described above in deionized water and then adjusting the pH of the dilution to about 3.5-4 using 0.1M sodium hydroxide solution; next, adding xanthan gum and glycerin to the solution and mixing the combination using a vortex stirrer (IKA® EUROSTAR Power Control from IKA Werke) for about an hour to ensure that an homogeneous gel is obtained.

A commercially available leave-in styling composition was used as comparative composition C3, which does not include xanthan gum, glycerin, or cyclodextrin. The main components of C3 are vitamins, cetearyl alcohol, preservatives, behentrimonium chloride, cetyl esters, vegetable extract, fragrance, amodimethicone, citric acid, vegetable oil, dyes, and water.

### Example 2—In Vitro Hair Swatch Performance Evaluation

2.1—Preparation of Treated Hair

The performance of composition 1A according to the present disclosure was tested on Caucasian curly swatches in comparison with comparative compositions C1, C2, C3 (benchmark), and deionized water (DI), as well as untreated hair.

To prepare the treated hair, curly hair swatches (Caucasian) were pre-washed with a standard shampoo (0.4 grams per gram of hair), and air dried overnight. The next day, the pre-washed hair swatches were washed once again with the standard shampoo. After the second wash, excess water was removed from the hair swatches and the hair swatches were ready to be treated with the compositions.

Each of compositions 1A and C1-C3, as well as DI water, were applied to three separate swatches of curly hair (15 swatches total) at a rate of about 0.2 grams of composition per gram of hair. The compositions were massaged through the hair for about one minute to ensure even distribution, and the treated hair was scrunched five times by hand. The swatches were then left to dry overnight in a chamber having a relative humidity of 20%. After overnight drying, the swatches were removed out from the chamber and were wrapped with thin paper, and then passed with fingers to break the film on the hair. After breaking the film, the hair swatches were subjected to evaluation. The hair swatches treated with water were used as untreated control, also so called “untreated” or “control” hair.

2.2—Evaluation of Treated Hair

Various properties of the above treated hair swatches were evaluated visually and/or by using instruments according to the following methods or protocols.

A. Evaluation of Frizz Control

Hair frizz control was measurement was carried out using an instrument system called HEAVEN (Hair Evaluation for Automated Volume Estimation) (Newton) (N=3). The system took pictures of hair swatches at a desired time point. The software acquired images of the hair swatches loaded inside the system equipped with a humidity and temperature control unit.

The treated hair swatches were placed in the HEAVEN system having 80% relative humidity (RH) at 27° C. for 24 hours, during which time the software acquired images of the swatches every hour. After 24 hours, the hair swatches were removed from the HEAVEN system.

B. Evaluation of Curl Definition

RUMBA is a polarization imaging system that takes successive images of a hair sample illuminated with infrared LED and under several polarization states, using a rotating polarizer. The images give a modulated intensity on each pixel. The modulation is caused by the birefringence of the hair fiber and contains the angle signature that can be extracted with the system's software to build an orientation image of the sample. The orientation data can be mapped onto a 2D image to visualize the whole sample. From this orientation data, a coefficient that relates to straightness of the hair can be defined—the straighter the hair fibers in the swatch, the closer to a single angle value the distribution of the orientation will be. The standard deviation of such a distribution is therefore inversely proportional to the perception of straightness. The same process is done for the alignment, where the alignment of a hair swatch is defined as the hair fibers being “locally straight.” Thus, a straightness coefficient is defined on a small scale of the 2D mapping of the orientation, which allows for a straightness coefficient to be obtained on every pixel of the image.

After the hair swatches were exposed to the condition of about 80% RH at 27° C. for about 24 hours and removed from the HEAVEN system, curl definitions of the hair swatches were analyzed using RUMBA (Orientation measurement for straightness & Alignment of hair fiber) from Bossa Nova Technologies, LLC and/or with photographic images. Each individual hair swatch was placed in the RUMBA and Rumba images were acquired for the hair swatches exposed to a condition of 80% relative humidity at 27° C. for 24 hours.

C. Differential Scanning calorimetry (DSC) Measurement

Differential Scanning calorimetry (DSC) measures the temperatures and heat flow associated with the thermal transition in a material. It is useful for investigating the structural characteristics of hair fibers. Keratin undergoes detectable transformations at various temperatures. Changes in these transformation temperatures can be used to estimate how a particular hair-treatment that may impact hair fibers. In this study, DSC was used to measure denaturation temperature (T_{d}). Denaturation temperature (T_{d}) has been used as a representation of the thermal stability of hair fibers, which is influenced, at least in part, by the cross-link density of the matrix (intermediate filament associated proteins, IFAP). Thermal stability (Ta) and its relationship in determining the thermal stability of hair fibers is established in the literature. Generally, the denaturation temperature of hair is higher if the hair strength/integrity is high and vice-versa.

The treated hair swatches were evaluated using DSC (DSC 2500 Discovery series from TA Instruments). To prepare hair samples prior to DSC analysis, 7-8 grams of each treated hair swatch was cut into particles with fine size (<0.5 mm). The hair particles were placed in a high-volume DSC pan chamber. About 50 μL of deionized water (DI water) was added into the pan and the hair particles were soaked in the water. The pan chamber was then sealed using a crimping dime. For each treated hair swatch samples, four pans were prepared (i.e. n=4).

The hair samples were equilibrated at room temperature for overnight. Approximately 24 hours later the hair samples were run on the DSC equipment starting from 40° C. and ramping by 10° C./min to 180° C.

T_{d} value was measured in degree Celsius, which represents the cross-linking density of the hair matrix. Trios' software was used to statistically analyze the denaturation temperature values measured by the DSC equipment. An increase in T_{d} indicates interaction of active and matrix hair protein.

D. Fiber Durability Testing (Cyclic Fatigue Tensile Testing, CFTT)

Cyclic fatigue testing is typically used to simulate the repeated tensile stress that occurs while combing and brushing. Cyclic fatigue data and survival probability, present both internal fiber flaws and external cuticle layer rupture. Cyclic fatigue testing provides a unique instrumental tool to demonstrate the progressive levels of impaired hair fibers. Fibers with relatively large flaws probably will break at lower numbers of cycles of applied stress than fibers with minor defects.

Fiber durability testing (cycles to break (CTB)) was carried out using a Cyclic Fatigue Tensile Tester (CFTT) 801 from Dia-Stron Ltd. The hair sample size for each swatch was about 50 fibers per sample. The fibers were 28 mm long. The experiment was carried out at about 23° C. and 45% relative humidity, and using a constant stress over a specified area.

The measured results were analyzed using UVWin analysis software vision 4.2.5.0 (n=50 fibers/sample). A higher CTB indicates a more durable fiber.

2.3—Results

A. HEAVEN Results

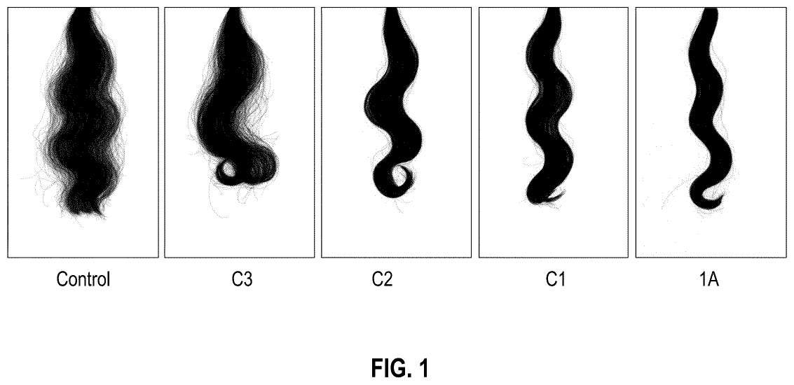

The HEAVEN images of the treated hair swatches after 24 hours of exposure to a condition of 80% relative humidity at 27° C. are shown in FIG. 1. As can be seen in the images, the hair swatch treated with composition 1 A appeared significantly less frizzy, compared to the hair treated with compositions C1, C2, and C3, as well as control hair treated with water. The effects were lasting, even after the hair was exposed to a condition of 80% relative humidity for 24 hours.

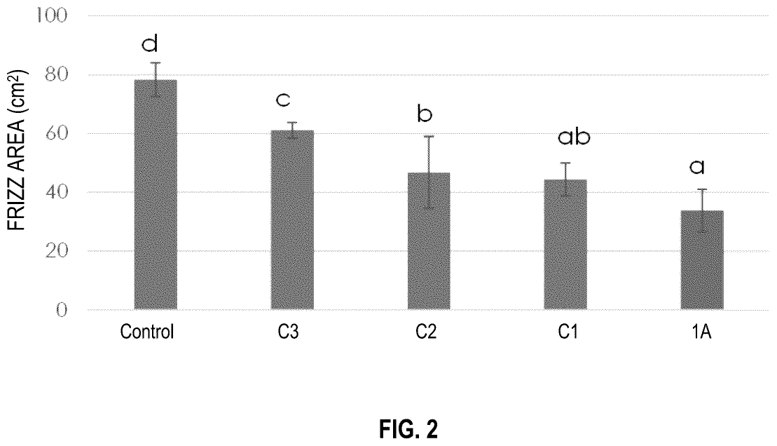

The statistic frizz levels of the treated hair swatches after 24 hours of exposure to a condition of 80% relative humidity at 27° C. were further illustrated in FIG. 2. As shown in FIG. 2, the hair treated with composition 1A had the least frizz, compared to the hair treated with compositions C1-C3 and the hair treated with water.

B. RUMBA Results

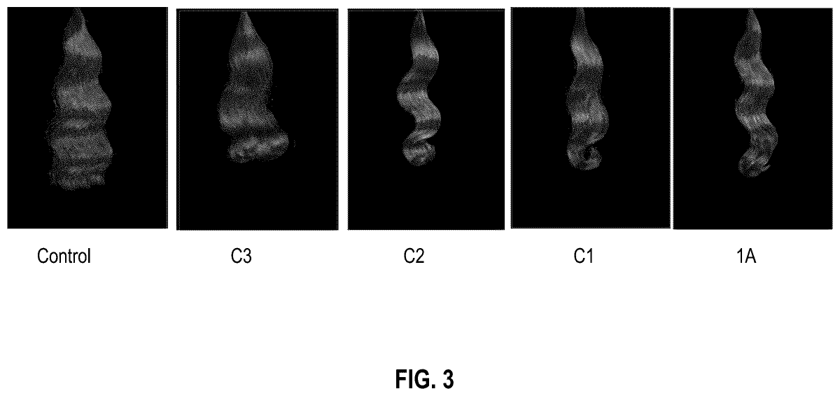

The 2D images obtained with the RUMBA system for the treated hair swatches after 24 hours of exposure to a condition of 80% relative humidity at 27° C. are shown in FIG. 3. As can be seen in FIG. 3, the hair treated with composition 1A demonstrated the best alignment of the hair, corresponding to the most defined curls and the best discipline, compared to the hair treated with compositions C1, C2, and C3, as well as the control hair treated with water.

C. DSC Results

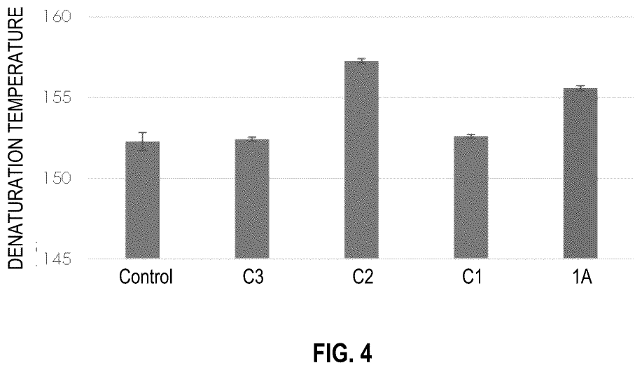

The denaturation temperatures of the treated hair measured using DSC are shown in the graph in FIG. 4. As can be seen, while the hair treated with composition C2 had the highest denaturation temperature, the hair treated with composition 1A had significantly higher denaturation temperature (Td) than the hair treated with compositions C1 and C3, as well as the untreated hair control. The results show that the integrity of the hair treated with composition 1 A was significantly improved.

D. Fiber Durability Results

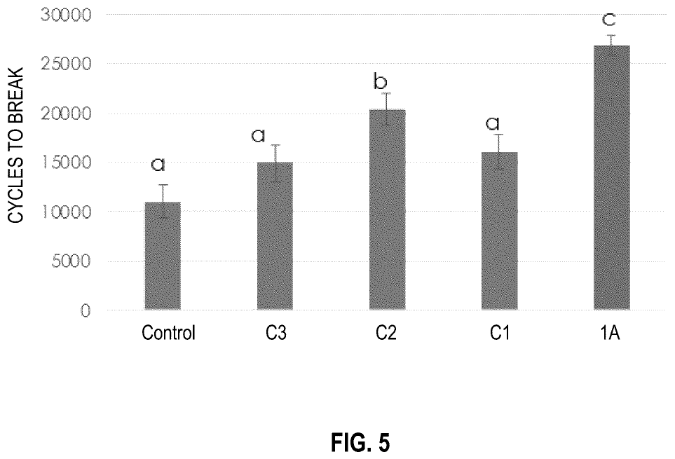

FIG. 5 is a graph showing the cycles needed to break the hair treated with composition 1A, compared to the cycles needed to break the hair treated with compositions C1-C3 and the control hair treated with water. As can be seen in FIG. 5, the cycles needed to break the hair treated with composition 1 A were significantly greater than the cycles needed to break the hair treated with compositions C1-C3 and the control hair treated with water.

E. Summary of Results

In sum, the above results show that the hair treated with composition 1A had overall significant improvement in frizz control, curl definition, x-linking density, and durability, compared to the untreated hair and the hair treated with compositions C1, C2, or C3. The curly hair treated with composition 1A was significantly less frizzy and had significantly higher durability compared to the hair treated with composition C2, which does not include polysaccharide thickening agents and polyols. In addition, The curly hair treated with composition 1A had significantly improved X-linking density and durability, compared to the curly hair treated with composition C1, where no carboxylic acids or salts thereof or cyclodextrins or derivatives thereof are included.

### Example 3—Split End Study

The performance of composition 1A according to the present disclosure on repairing split ends of hair was evaluated on curly hair swatches in comparison with comparative compositions C1 and C2, as well as untreated control.

The study was performed according to the following procedure:
1. Wash hair swatches with a standard shampoo (0.4 g/g hair).
2. Blots dry the hair swatches with paper towels.
3. Place the wet hair swatches in a weighing boat.
4. Apply each composition 1A, C1, and C2, as well as water, in drop on a separate hair swatch using a plastic pipette from top to bottom, where DI water and composition C2 were applied in a ratio of about 0.4 grams of composition per gram of hair, and compositions C1 and 1 A were applied in a ratio of about 0.2 grams of composition per gram of hair.
5. Next, hold each swatch, and massage 1 min with fingers to make sure product was evenly distributed.
6. Air dry the treated hair swatches overnight.
7. On the next day, break the crust/film on the dried hair by passing the hair with fingers.
8. Count completely sealed split end fiber.
9. Wash the hair swatches using shampoo three times (Shampoo 3×).
10. Count post shampoo sealed split end fiber.

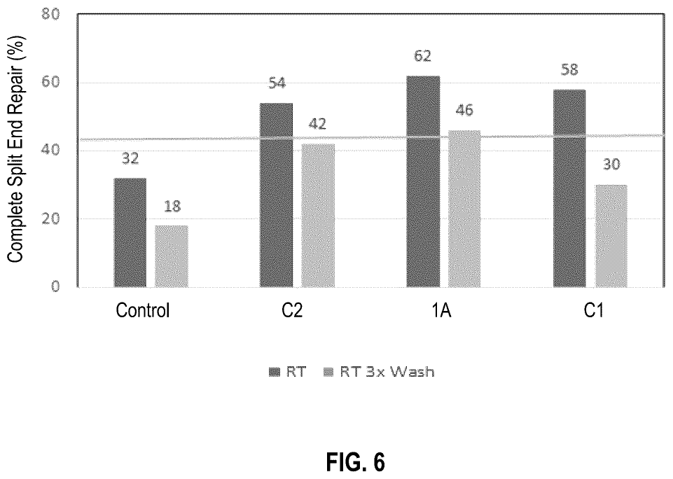

The results of the split ends study are illustrated in FIG. 6, which shows the percentage of complete split end repair achieved by composition 1A in comparison with composition C1 and C2, as well as untreated control, counted on the next day of the application when the hair was dry and after breaking the film and post three times of shampooing after the treatment using the compositions. As shown in FIG. 6, the control hair, i.e., the hair treated with water, had the most split ends. The hair treated with compositions 1A, C1, and C2 all show significantly improved split end repair compared to the control after the hair swatches were treated with the compositions and before the hair was shampooed. After the hair swatches treated with each composition and the untreated hair swatch were washed three times using a shampoo, the complete split end repair percentages in all hair swatches decreased. However, compared to the untreated hair, the hair ends of the hair treated with composition 1 A were still significantly repaired, and the repair performance of composition 1A was still better than composition C2 and C1. As such, the split end repair benefits provided by composition 1A is lasting, even after the hair was shampooed three times.

### Example 4—Treatment Routine Study

A hair treatment routine involving rinsing off the composition according to the present disclosure from treated hair at the end of the treatment to eliminate crunchiness of the treated hair was studied.

In this study, two curly hair swatches (Caucasian) were pre-washed with a standard shampoo (0.4 g/g hair). After the shampoo was rinsed off, excess water was removed from the hair swatches. Composition 1A was then applied to one pre-washed hair swatch in a bath ratio of about 0.2 g of composition per gram of hair while the hair was still wet or damp. The hair treated with composition 1A was thoroughly massaged for about one (1) minute to ensure that the composition was evenly distributed on the hair. While composition 1 A was left on the hair, a commercially available detangling conditioner was applied to the hair and the hair was massaged for about 1 minute to ensure even distribution of the conditioner on the entire hair swatch. Next, the hair swatch was rinsed to remove composition 1A and the conditioner, and the swatch was then scrunched five times by hand and subsequently air-dried. As a control, the other pre-washed hair swatch was treated according to the same routine process but no composition 1 A was applied to the hair. After the hair was dried, both of the hair swatches were evaluated by tactile assessment and visual observation.

The evaluation revealed that the hair treated with composition 1A according to the rinse-off routine, where the hair was rinsed at the end of the treatment routine, felt smooth and not crunchy. In addition, compared to the control hair swatch not treated with composition 1A, the hair treated with composition 1A according to the rinse-off routine had better curl definition and less frizzy.

### Example 5—In Vivo Testing

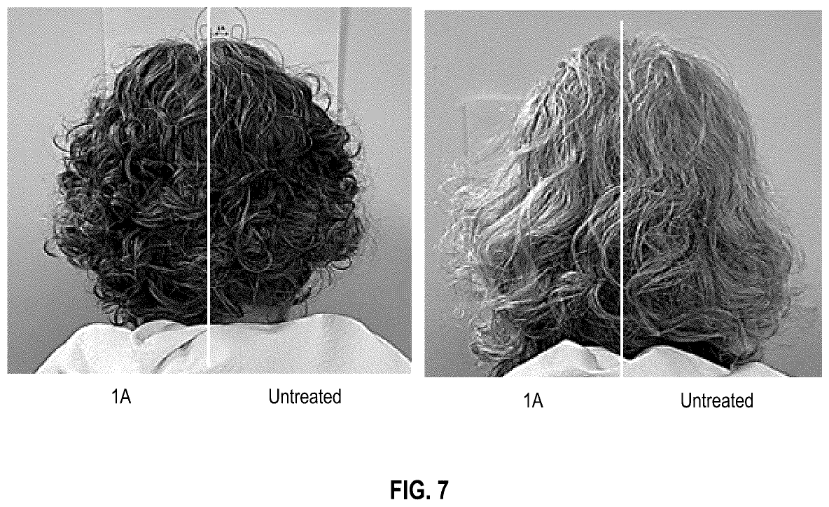

The performance of composition 1A was tested on consumers. About 10 grams of composition 1A was applied on the consumers' hair via half-head application. Twenty-four hours after the application, the hair was assessed for frizz control and curl definition benefits. FIG. 7 shows photographs of the hair of two consumers 24 hours after the treatment. The left side of head was treated with composition 1A, and the right side of the head was untreated control, i.e., treated with water. As can be seen in FIG. 7, composition 1 A provides noticeable benefits such as curl definition, discipline, style/shape hold, and volume and density control to the hair, wherein the hair varied in volume, curl degree, and color.

### Example 6—Progressive Benefits Evaluation

The performance of composition 1A via multiple applications was evaluated to hair swatches that were bleached twice, compared to multiple applications to the same type of hair swatches that were natural, i.e., not bleached.

In this study, unbleached natural hair swatches and hair swatches that were bleached twice (pre-bleached hair) were treated with composition 1A in a bath ratio of about 0.2 grams of composition per gram of hair. After application of the composition, the hair swatches were massaged for about 1 minute with fingers to ensure that the composition was evenly distributed on the hair, and then air dried overnight. The next day the denaturation temperatures (T_{d}) of the hair swatches were measured using DSC (DSC 2500 Discovery series from TA Instruments) according to the process described above.

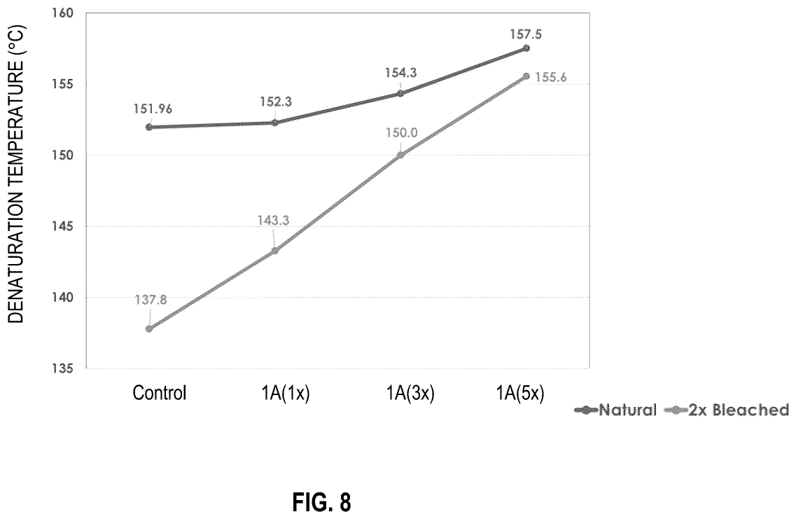

The measurement results were analyzed and shown by the graph in FIG. 8. As can be seen in FIG. 8, after bleaching, the denaturation temperature of the unbleached natural hair significantly decreased, indicating that the hair was damaged due to bleaching. After being treated with composition 1A once, three times, or five times, the denaturation temperatures of the natural hair and the bleached hair both increased, the more times of treatment, the higher the denaturation temperature. The results demonstrate that the treatment using composition 1A improved the integrity of both the natural hair (top line) and the bleached hair (bottom line), i.e., damaged hair. In addition, the improvement of integrity provided to the damaged hair was more significant, compared to the improvement provided to the natural hair. For example, as shown in FIG. 8, after the bleached hair was treated with composition 1A five times, the denaturation temperature of the bleached hair significantly increased to a level that was close to that of the natural hair after the natural hair was treated with composition 1 A five times, and was even higher than the denaturation temperature of the untreated natural hair.

### Example 7—Evaluation of Lastingness of Benefits

A study was undertaken to evaluate the lastingness of benefits imparted to hair treated with compositions according to the disclosure, using the compositions in Tables 2-1 and 2-2.

TABLE 2-1
Compositions According to the Disclosure

| | 2A | 2B | 2C |
|---|---|---|---|
| XANTHAN GUM | 1 | 1 | |
| GUAR GUM | | | 1 |
| GLYCERIN | 1 | 1 | 1 |
| CITRIC ACID | 2.5 | 2.5 | 2.5 |
| β-CYCLODEXTRIN | 1.5 | 1.5 | 1.5 |
| SOYBEAN OIL | | 1 | |
| PEG-40 HYDROGENATED | | | 1 |
| CASTOR OIL | | | |
| WATER | QS to 100 | QS to 100 | QS to 100 |

TABLE 2-2
Comparative Compositions

| | C4 | C5 |
|---|---|---|
| TARTARIC ACID | 1.2 | |
| LACTIC ACID | 0.36 | |
| ARGININE | 1 | |
| TRIETHANOLAMINE | 0.7 | |
| SODIUM BENZOATE | 0.3 | |
| CETYL ESTERS (and) CETYL ESTERS | 1 | 1 |
| CETEARYL ALCOHOL | 6 | 5 |
| BEHENTRIMONIUM CHLORIDE | 3 | 1 |
| COPAIFERA OFFICINALIS (BALSAM | 0.17 | |
| COPAIBA) RESIN | | |
| COCOS NUCIFERA (COCONUT) OIL | 4.3 | |
| KERATIN COMPLEX | | 0.5 |
| ARGAN OIL | | 0.1 |
| HYDROXYPROPYLTRIMONIUM | | 0.01 |
| LEMON PROTEIN | | |
| PROPYLENE GLYCOL | 1.3 | |
| DICAPRYLYL CARBONATE | 1 | |
| TOCOPHEROL | | 0.01 |
| AMODIMETHICONE (and) | | 2.4 |
| TRIDECETH-6 (and) CETRIMONIUM | | |
| CHLORIDE | | |
| ADDITIVES (fragrance, preservatives, | <2 | <2 |
| pigments, etc.) | | |
| WATER | QS to 100 | QS to 100 |

In order to evaluate lastingness of frizz control and curl definition, each of compositions 2A-2C or C4-05 was applied to three separate pre-washed swatches of medium-curly hair at a rate of 0.4 grams of product per gram of hair. The swatches were placed in a controlled chamber (80% humidity, 23° C.) and left for 24 hours. Three additional pre-washed swatches of medium-curly hair with no product applied were also placed in the chamber as a control.

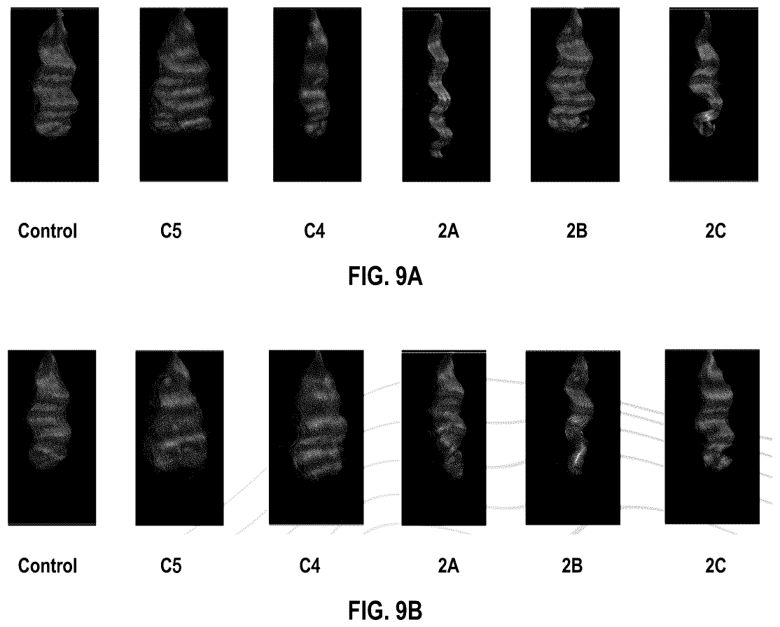

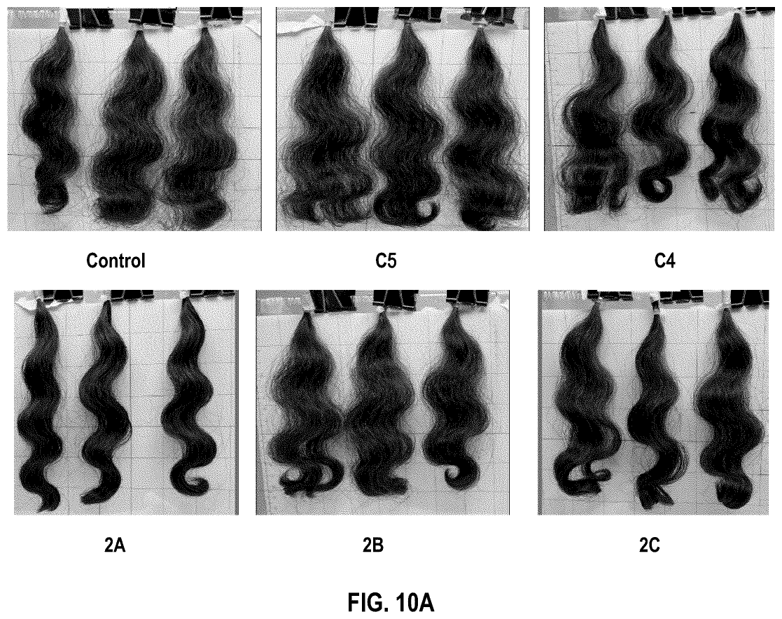

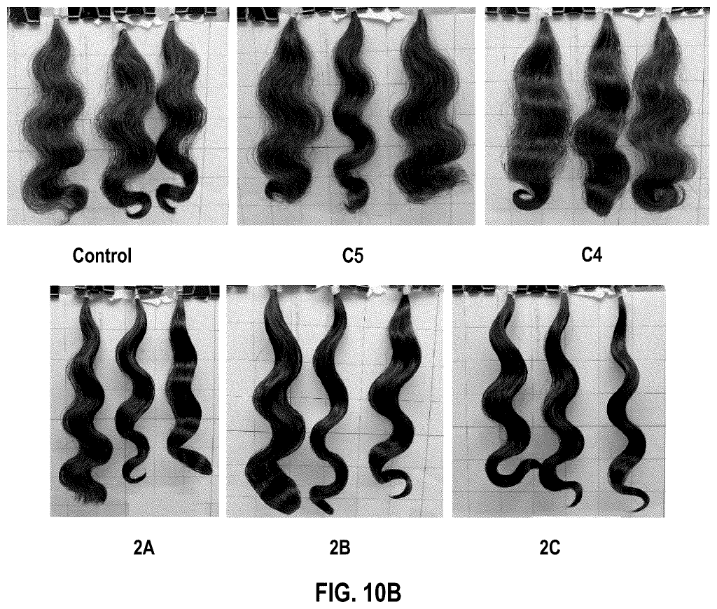

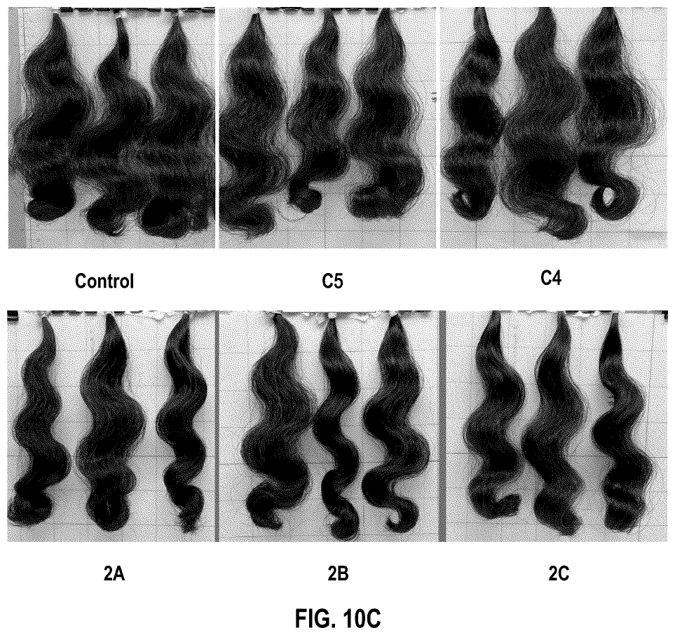

After 24 hours, the swatches (18 total) were removed from the chamber (time =T₀) and analyzed via the Rumba protocol described above. The 2D images obtained at T₀ for one swatch treated with each of compositions 2A-2C, C4-05, and the control are shown in FIG. 9A. Photographs of the treated swatches at T₀ are shown in FIG. 10A. As can be seen in FIGS. 9A and 10A, the hair treated with composition 2A demonstrated the best alignment of the hair, corresponding to the most defined curls and the best discipline, immediately after treatment and before the hair was washed.

Next, the swatches were washed with a standard shampoo, rinsed, wrung dry, and left to equilibrate at room temperature overnight. This wash-rinse-equilibrate cycle (which is considered to correspond to a typical consumer's daily shampoo routine) was repeated five more times. After the third cycle, photographs of the swatches were again taken, and are seen in FIG. 10B. As FIG. 10B shows, after 3 wash-rinse-equilibrate cycles, the swatches treated with compositions 2A-2C show significantly better frizz control and curl retention than the swatches treated with compositions C4-05 and the control swatches.

After the sixth cycle, photographs of the swatches were again taken, and are shown in FIG. 10C. As can be seen in FIG. 10C, the swatches treated with compositions 2A-2C show significantly better frizz control and curl retention than the swatches treated with compositions C4-05 and the control swatches, even after six wash-rinse-equilibrate cycles.

Next, the swatches were again placed in the controlled chamber (80% humidity, 23° C.) and left for 24 hours. After 24 hours, the swatches were removed from the chamber (time=T₁) and again analyzed via the Rumba protocol. The 2D images obtained at T₁ for one swatch treated with each of compositions 2A-2C, C4-C5, and the control are shown in FIG. 9B. Photographs of the treated swatches at T₁ are shown in FIG. 10C. As can be seen in FIGS. 9B and 10C, hair that has been treated with compositions 2A-2C demonstrates significantly better frizz control and curl definition after undergoing two high-humidity exposures and six wash-rinse-equilibrate cycles, compared to hair treated with compositions C4-C5 and the control.

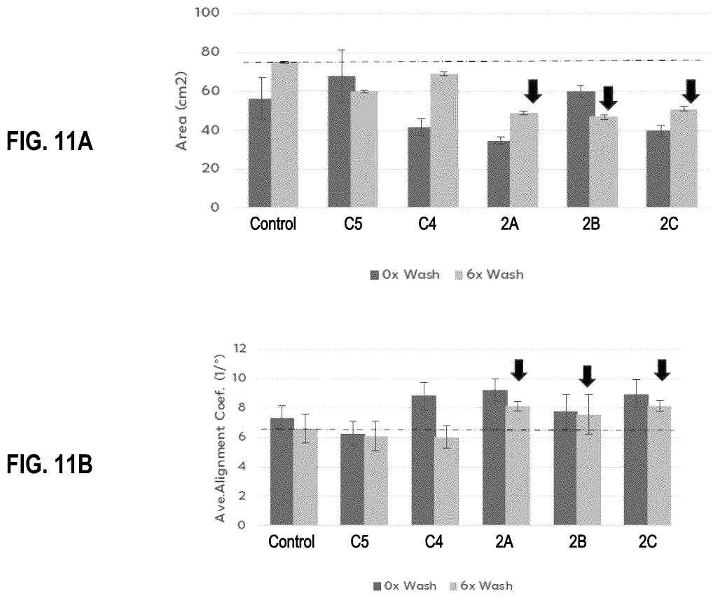

FIGS. 11A-11B are graphical representations comparing the frizz control (FIG. 11A) and curl definition (FIG. 11B) of the swatches at T₀ (0× wash) and T₁ (6× wash). In FIG. 11A, a lower area value shows better frizz control, and in FIG. 11B, a higher value for average alignment coefficient shows better curl definition. As shown, FIGS. 11A and 11B confirm that hair that has been treated with compositions 2A-2C surprisingly demonstrates significantly better frizz control and curl definition after undergoing two high-humidity exposures and six wash-rinse-equilibrate cycles, than hair treated with compositions C4-C5 and the control.

### Example 8—Additional Compositions

The compositions in Table 3, which represent additional formulations for treating or styling hair according to embodiments of the disclosure, can be prepared and used as described herein.

TABLE 3

| | Additional Compositions | | |
|---|---|---|---|
| | 3A | 3B | 3C |
| HYDROXYETHYLCELLULOSE | 0.25 | | |
| GUAR GUM | | 4.5 | 1.5 |
| GLYCERIN | 0.25 | | |
| ETHYLENE GLYCOL | | 3.5 | 1 |
| MALEIC ACID | 0.5 | | 2 |
| LACTIC ACID | | 6 | 1 |
| CYCLODEXTRIN | 0.5 | 6 | 2.5 |
| ADDITIONAL COMPOUNDS (pH | ≤1 | ≤5 | ≤3 |
| adjusters, preservatives, plant | | | |
| extracts, vitamins, dyes, etc.) | | | |
| WATER | QS to 100 | QS to 100 | QS to 100 |

The above Examples demonstrate that compositions according to the disclosure surprisingly provide improved curl definition and/or frizz control to hair, which lasts over time even after exposure to high humidity and even after shampooing the hair, over a range of different mole ratios, concentrations, and components.

It will be apparent to those skilled in the art that various modifications and variations can be made in the compositions and methods according to the disclosure without departing from the spirit or scope of the disclosure. Thus, it is intended that the disclosure cover such modifications and variations and their equivalents.

1-34. (canceled)

35. A composition for treating keratin fibers, comprising:
(a) at least one carboxylic acid or salt thereof;
(b) at least one cyclodextrin or derivative thereof;
(c) at least one polysaccharide thickening agent;
(d) at least one polyol; and
(e) water;
wherein the composition has a pH of less than 7.

36. The composition according to claim 35, wherein the composition has a mole ratio of (a) carboxylic acids and salts thereof to (b) cyclodextrins and derivatives thereof ranging from about 0.5:1 to about 25:3.

37. The composition according to claim 35, wherein the composition has a weight ratio of the total amount of (a) carboxylic acids and salts thereof to (b) cyclodextrins and derivatives thereof ranging from about 1:3 to about 6:1.

38. The composition according to claim 35, wherein the at least one carboxylic acid or salt thereof is chosen from mono-carboxylic acids, di-carboxylic acids, tri-carboxylic acids, salts thereof, or mixtures of two or more thereof.

39. The composition according to claim 35, wherein the at least one carboxylic acid, salts thereof, or mixtures of two or more thereof is chosen from citric acid, lactic acid, malic acid, maleic acid, tartaric acid, glycolic acid, phytic acid, salts thereof, or mixtures of two or more thereof.

40. The composition according to claim 35, wherein the total amount of carboxylic acids and salts thereof ranges from about 1% to about 20% by weight, relative to the total weight of the composition.

41. The composition according to claim 35, wherein the at least one cyclodextrin or derivative thereof is chosen from compounds of formula: 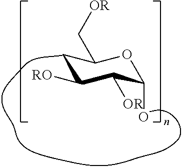
wherein:
R is chosen from H, CH₃, or a hydroxypropyl group; and
n is an integer ranging from 6 to 8.

42. The composition according to claim 35, wherein the at least one cyclodextrin or derivative thereof is chosen from α-cyclodextrin, β-cyclodextrin, γ-cyclodextrin, methyl derivatives of α-cyclodextrin, methyl derivatives of β-cyclodextrin, methyl derivatives of γ-cyclodextrin, hydroxypropyl derivatives of α-cyclodextrin, hydroxypropyl derivatives of β-cyclodextrin, hydroxypropyl derivatives of γ-cyclodextrin, or mixtures of two or more thereof.

43. The composition according to claim 35, wherein the total amount of cyclodextrins and derivatives thereof ranges from about 0.01% to about 20% by weight, relative to the total weight of the composition.

44. The composition according to claim 35, wherein the at least one polysaccharide thickening agent is chosen from inulins, gums, celluloses, starches, or mixtures of two or more thereof.

45. The composition according to claim 35, wherein the at least one polysaccharide thickening agent is chosen from gum arabic, tragacanth gum, karaya gum, guar gum, gellan gum, tara gum, locust bean gum, tamarind gum, xanthan gum, dehydroxanthan gum, Seneca gum, sclerotium gum, gellan gum, or mixtures of two or more thereof.

46. The composition according to claim 35, wherein the total amount of polysaccharide thickening agents ranges from 0.05% to about 5% by weight, relative to the total weight of the composition.

47. The composition according to claim 35, wherein the at least one polyol is chosen from ethylene glycol, propylene glycol, butylene glycol, hexylene glycol, pentylene glycol, diethylene glycol, dipropylene glycol, 1,3 propanediol, glycerin, polyethylene glycols, or mixtures of two or more thereof.

48. The composition according to claim 35, wherein the total amount of polyols is at least 0.5% by weight, relative to the total weight of the composition.

49. A composition for treating curly hair comprising:
(a) at least one carboxylic acid chosen from citric acid and/or salts thereof, wherein the total amount of citric acid and salts thereof ranges from about 0.1% to about 20%;
(b) at least one cyclodextrin chosen from β-cyclodextrin or derivatives thereof, wherein the total amount of β-cyclodextrin and derivatives thereof ranges from about 0.01% to about 20%;
(c) at least one polysaccharide thickening agent chosen from gums, wherein the total amount of polysaccharide thickening agents ranges from about 0.05% to about 5%;
(d) at least one polyol, wherein the total amount of polyols is at least 0.5%; and
(e) water,
wherein the composition has a pH ranging from about 2 to about 6, and
wherein all amounts are by weight, relative to the total weight of the composition.

50. A method for treating hair, comprising applying to the hair a composition comprising:
(a) at least one carboxylic acid or salt thereof;
(b) at least one cyclodextrin or derivative thereof;
(c) at least one polysaccharide thickening agent;
(d) at least one polyol; and
(e) water;
wherein the composition has a pH of less than 7.

51. The method of claim 50, further comprising heating the hair at a temperature ranging from about 50° C. to about 225° C.

52. The method of claim 51, wherein the hair is heated for a period of time ranging from about 1 minute to about 5 minutes.

53. The method according to claim 51, wherein the composition comprises:
(a) citric acid and/or a salt thereof;
(b) β-cyclodextrin and/or a derivative thereof;
(c) at least one polysaccharide thickening agent chosen from gums;
(d) at least one polyol; and
(e) water;
wherein the composition has a pH ranging from about 2 to about 6.

54. The method according to claim 53, wherein the composition has a mole ratio of (a) carboxylic acids and salts thereof to (b) cyclodextrins and derivatives thereof ranging from about 0.5:1 to about 25:3, and/or a weight ratio of the total amount of (a) carboxylic acids and salts thereof to (b) cyclodextrins and derivatives thereof ranging from about 1:3 to about 6:1.
